(19) United States
(12) Patent Application Publication
(10) Pub. No.: US 2009/0162305 A1
(43) Pub. Date: Jun. 25, 2009

(54) FORMULATIONS OF LOW OIL CONTENT COMPRISING DIPHENYLMETHANE DERIVATIVES

(75) Inventors: Gabriele Vielhaber, Holzminden (DE); Gerhard Schmaus, Höxter (DE); Sabine Lange, Holzminden (DE); Karin Schaper, Linnenkamp (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzminden (DE)

(21) Appl. No.: 12/159,886

(22) PCT Filed: Jan. 5, 2007
(86) PCT No.: PCT/EP07/50125

§ 371 Date: Oct. 16, 2008

Related U.S. Application Data
(60) Provisional application No. 60/756,205, filed on Jan. 5, 2006.

(51) Int. Cl. A61K 8/34; A61K 31/05; A61Q 19/02

(52) U.S. Cl. 424/62; 514/736

(57) ABSTRACT

The present invention relates to specific (cosmetic) formulations for improving the bioavailability and activity of skin- or hair-lightening or senile keratosis-reducing diphenylmethane derivatives (tyrosinase inhibitors) of the following formula 1:

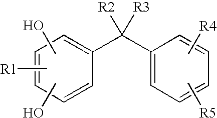

### CROSS-REFERENCE TO RELATED APPLICATIONS

This application claims benefit of priority to PCT/EP2007/050125, filed on Jan. 5, 2007, which asserts priority to U.S. Provisional Application No. 60/56,205, filed on Jan. 5, 2006, which are incorporated herein by reference in their entireties.

The present invention relates to specific (cosmetic) formulations for improving the bioavailability and activity of skin- or hair-lightening or senile keratosis-reducing diphenylmethane derivatives (tyrosinase inhibitors) of the following formula 1:

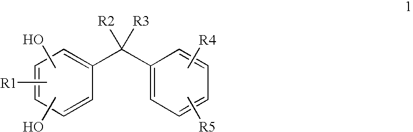

wherein:

### R1 is

hydrogen,
methyl,
straight-chain or branched, saturated or unsaturated alkyl having 2-4 C atoms,
OH or
halogen,

### R2 is

hydrogen,
methyl or
straight-chain or branched, saturated or unsaturated alkyl having 2-5 C atoms,

### R3 is

methyl or
straight-chain or branched, saturated or unsaturated alkyl having 2-5 C atoms,
   and
   R4 and R5 are, independently of one another,
hydrogen,
methyl,
straight-chain or branched, saturated or unsaturated alkyl having 2-5 C atoms,
OH or
halogen.

In this context, the substituents OH, R1, R4 and R5 can in each case occupy (as indicated by the drawing) any desired position on the particular aromatic ring (ortho, meta or para to the bridge between the rings).

In the field of the cosmetics industry, there is an increasing demand for agents for lightening the skin and hair and for agents for combating senile keratosis. In this context, cosmetics for lightening the skin and hair and for combating senile keratosis play a large role above all in Asian countries with a dark-skinned and dark-haired population, but agents for such cosmetic treatments are also gaining in importance in the central European region and in the USA.

The skin and hair colour of humans is substantially determined via the number of melanocytes, and via the melanin concentration and the intensity of melanin biosynthesis, on the one hand intrinsic factors, such as the genetic make-up of an individual, and on the other hand extrinsic factors, such as, in particular, the intensity and frequency of exposure to UV, having a significant influence on skin and hair colour.

Skin- and hair-lightening active compounds conventionally intervene in melanin metabolism or catabolism. The melanin pigments, which as a rule are brown to black in colour, are formed in the melanocytes of the skin, transferred into the keratinocytes and cause the colouration of the skin or hair. The brown-black eumelanins are chiefly formed in mammals from hydroxy-substituted aromatic amino acids, such as L-tyrosine and L-DOPA, and the yellow to red phaeomelanins are additionally formed from sulfur-containing molecules (Cosmetics & Toiletries 1996, 111 (5), 43-51). Starting from L-tyrosine, L-3,4-dihydroxyphenylalanine (L-DOPA) is formed by the copper-containing key enzyme tyrosinase, and is in turn converted into dopachrome by tyrosinase. The latter is oxidized to melanin via several steps catalysed by various enzymes.

Skin- and hair-lightening agents are used for various reasons. If the melanin-forming melanocytes are not distributed uniformly in the human skin for whatever reason, pigmental moles which are either lighter or darker than the surrounding areas of skin arise. To eliminate this problem, lightening agents which at least partly help to compensate such pigmental moles are employed. In addition, for many people there is the need to lightening their naturally dark skin colour or to prevent pigmentation of the skin. Very safe and effective skin- and hair-lightening agents are necessary for this. Many skin- and hair-lightening compositions comprise more or less potent tyrosinase inhibitors. However, only one possible route to lightening the skin and hair is taken by this means.

UV-absorbing substances are occasionally also employed for protection against the increase in skin pigmentation induced by UV light. However, this is an effect of purely physical origin and therefore differs from the biological action of skin-lightening agents on cellular melanin formation, which is also detectable in the absence of UV light. In fact, only the UV-induced browning of skin can be prevented by UV filters, whereas a lightening of the skin can also be brought about with biologically active skin lighteners which intervene in melanin biosynthesis.

Hydroquinone, hydroquinone derivatives, such as e.g. arbutin, vitamin C, derivatives of ascorbic acid, such as e.g. ascorbyl palmitate, kojic acid and derivatives of kojic acid, such as e.g. kojic acid dipalmitate, are used in particular in commercially available skin- and hair-lightening compositions.

One of the most frequently used skin- and hair-lightening agents is hydroquinone. However, the substance has a cytotoxic effect on melanocytes and irritates the skin. Such preparations are therefore no longer approved for cosmetic uses e.g. in Europe, Japan and South Africa. Furthermore, hydroquinone is very sensitive to oxidation and can be stabilized in cosmetic formulations only with difficulty.

Vitamin C and ascorbic acid derivatives have only an inadequate action on the skin. They furthermore do not act directly as tyrosinase inhibitors, but reduce the coloured intermediate stages of melanin biosynthesis.

Kojic acid (5-hydroxy-2-hydroxymethyl-4-pyranone) is a tyrosinase inhibitor which, via a chelating of the copper atoms of the enzyme, inhibits the catalytic action thereof; it is employed in commercial skin- and hair-lightening compositions, but has a high sensitizing potential and causes contact allergies.

In the search for novel agents which have a skin- and hair-lightening action and/or are active against senile keratosis, efforts are accordingly being made quite generally to discover substances which inhibit the enzyme tyrosinase in the lowest possible concentration, whereby it is furthermore to be ensured that these substances used in cosmetic and/or pharmaceutical products, in addition to having a high activity at the lowest possible concentrations, must also additionally be
toxicologically acceptable,
readily tolerated by the skin,
heat-stable (in particular in the conventional cosmetic and/or pharmaceutical formulations),
preferably odourless and
inexpensive to prepare (i.e. employing standard processes and/or starting from standard precursors).

The search for suitable (active) substances which have one or more of the properties mentioned to an adequate extent is made difficult for the person skilled in the art in that there is no clear dependency between the chemical structure of a substance on the one hand and its biological activity and its stability on the other hand. Furthermore, there is no predictable connection between the skin- and hair-lightening action, the toxicological acceptability, the skin tolerability and/or the stability of potential active compounds. A particular prerequisite for the use of an active substance in practice is moreover its stability towards chemical substances which are conventionally used as concomitant substances in cosmetics and towards (sun- or UV) light.

As described in detail in WO 2004/105736, diphenylmethane derivatives of the formula 1 in particular meet the abovementioned product requirements in an ideal manner. As is furthermore described in WO 2004/105736, in this context the skin- and hair-lightening and senile keratosis-reducing activity of substances of the formula 1 is based chiefly on the inhibition of tyrosinase, a key enzyme in melanin formation. It has been possible to demonstrate this clearly by appropriate in vitro experiments, such as, inter alia, enzyme assays with fungal tyrosinase and cell biology studies on B16 mouse melanoma cells.

Nevertheless, a very good tyrosinase-inhibiting activity in such in vitro experiments must not necessarily also mean an outstanding activity during later use in cosmetic products. A prerequisite of achieving an adequate skin- and hair-lightening or an adequate reduction in senile keratosis in the human in vivo situation is moreover also a very good bioavailability of the active compound at the actual site of action. In the case of skin-lightening agents, in this context the active compound must penetrate into deeper layers of the epidermis and enter into the melanocytes located there, in order to be able to display its activity.

The object of the invention was therefore to provide formulations having a potent skin- and/or hair-lightening and/or senile keratosis-reducing activity at the lowest possible dose of the skin- and/or hair-lightening active compound(s). The object is achieved according to the invention by a formulation having a low content of an oily phase in the total formulation, in particular a formulation according to claim 1.

Surprisingly, our own comparative human in vivo studies with formulations having a varying content of oily phases have shown that, in particular, formulations having a high content of hydrophilic components and a greatly reduced content of the oily phase achieve these objects particularly well and can thus be particularly preferably employed as transport systems for tyrosinase-inhibiting agents of the formula 1.

A significantly more potent skin- and hair-lightening and senile keratosis-reducing activity and a significantly higher bioavailability was found in particular for a formulation according to the invention in which the content of the oily phase is in the range of from 0.05 to 12 wt. %, based on the total weight of the formulation.

Preferred embodiments of the formulations which are preferred according to the invention and uses thereof are described in the following and in the examples and the claims.

In this context, diphenylmethane derivatives of the formula 1, preferably compounds of the formula 2, wherein R1 and R3 have the abovementioned meaning, and in particular the styrylresorcinol of the formula 3 described in more detail in the following (CARN: 85-27-8; 4-(1-phenylethyl)-1,3-dihydroxybenzene) can be incorporated without problems into formulations according to the invention.

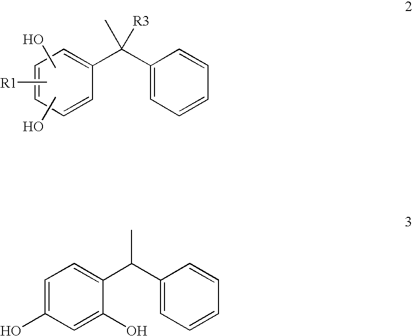

Diphenylmethane derivatives of the formula 1, preferably compounds of the formula 2, and in particular the styrylresorcinol of the formula 3 described in more detail in the following, are released in an improved manner from the formulations according to the invention when used on skin and/or hair, and show an improved skin- and hair-lightening and an improved senile keratosis-reducing action.

The particularly suitable formulations according to the invention having a low content of oily phase for inhibition of tyrosinase are chiefly used according to the invention for cosmetic reasons, but in exceptional cases they can also have a therapeutic character.

U.S. Pat. No. 5,399,785 describes some tetrahydroxy compounds which fall under the above formula 1 if R4 and R5 each denote OH. It is indeed stated generally in U.S. Pat. No. 5,399,785 that the compounds described there can be incorporated into cosmetic formulations, but no reference to specific cosmetic formulations having a defined content of an oily phase is to be found.

In this context, the concentration of the diphenylmethane derivatives of the formula 1 in the formulations, in particular to be applied topically, according to the invention is preferably in the range of from 0.001 to 6 wt. %, preferably in the range of from 0.01 to 4 wt. % and particularly preferably in the range of from 0.01 to 2 wt. %. The tyrosinase-inhibiting active compound can be employed here (a) prophylactically or (b) as required.

The concentration of the amount of active compound to be applied e.g. daily varies and depends on the physiological state of the subject and individual-specific parameters, such as age or body weight. Diphenylmethane derivatives of the formula 1 can be employed in the formulations according to the invention by themselves, as mixtures or also in combination with further tyrosinase-inhibiting substances.

It is to be pointed out that the term diphenylmethane derivatives in the context of the present text also includes, in the case of the derivatives of the formula 1 which have differently substituted phenyl radicals and for which at the same time R2 and R3 are different, the pure S-configured enantiomers, the R-configured enantiomers and any desired mixtures of S- and R-configured enantiomers. For commercial reasons, it is indeed particularly advantageous in these cases to employ mixtures of racemates of the particular diphenylmethane derivatives for lightening skin and/or for combating senile keratosis, since these are particularly readily accessible by synthesis, but the pure enantiomers or non-racemic mixtures of these enantiomers are likewise suitable for the purposes according to the invention.

In this context, the diphenylmethane derivatives of the formula 1 used according to the invention can be incorporated without difficulties into the chiefly aqueous cosmetic or dermatological formulations according to the invention, such as, inter alia, pump sprays, aerosol sprays, creams, ointments, tinctures, lotions and specific nail care products and the like. It is also possible here, and in some cases advantageous, to combine diphenylmethane derivatives of the formula 1 with further active compounds, for example with other substances having a skin- and hair-lightening action or which act against senile keratosis, in the formulations according to the invention. The cosmetic and/or dermatological/keratological formulations according to the invention comprising diphenylmethane derivatives of the formula 1 and having a reduced content of oily phase can otherwise have the conventional composition here and serve for the treatment of skin and/or hair in the sense of a dermatological or keratological treatment or a treatment in the sense of care cosmetics. However, they can also be employed in decorative cosmetics.

The significant more potent activity and significantly higher bioavailability has been found for formulations according to the invention in which the content of the oily phase is from 0.05 to 12 wt. %, preferably from 0.1 to 10 wt. % and particularly preferably 0.5-8 wt. %, based on the total weight of the formulation. An improved skin-lightening activity has been found, in particular, for the formulations according to the invention (comprising an oily phase in the preferred ranges of amounts).

So that an improved activity and bioavailability of the diphenylmethane derivatives of the formula 1 from a cosmetic formulation can be achieved, a content of the oily phase in the broader or narrower sense of greater than 12 wt. %, based on the total weight of the formulation, is to be avoided.

In this context, for the preparation of a formulation according to the invention it is irrelevant whether the substances of the oily phase are processed (partly) together, separately or in a mixture with other, optionally water-soluble, constituents of the formulation.

An oily phase in the broader sense of the present invention includes the following substance groups:
(i) straight- or branched-chain saturated paraffins (mineral oils) having at least 15 C atoms, in particular having 18 to 45 C atoms;
(ii) esters having at least 12 C atoms of straight- or branched-chain saturated or unsaturated fatty acids having 6 to 30 C atoms and straight- or branched-chain saturated or unsaturated mono-, di- or triols having 3 to 30 C atoms, these esters containing no free hydroxyl groups;
(iii) esters of benzoic acid and straight- or branched-chain saturated or unsaturated monoalkanols having 8 to 20 C atoms;
(iv) mono- or diesters of alcohols having 3 to 30 C atoms and naphthalene-mono-or-dicarboxylic acids, in particular naphthalenemonocarboxylic acid C6-C18 esters and naphthalenedicarboxylic acid di-C6-C18 esters;
(v) straight- or branched-chain saturated di-C6-C18-alkyl ethers;
(vi) silicone oils;
(vii) 2-alkyl-1-alkanols of the formula (VII)

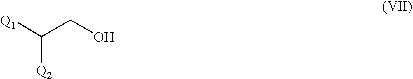

wherein
Q1 denotes an alkyl radical having 6 to 24 C atoms and
Q2 denotes an alkyl radical having 4 to 16 C atoms.

According to the invention, the content of these substances in a formulation according to the invention is limited to the amounts described.

An oily phase in the narrower (and preferred) sense of the present invention. i.e. the substances present, according to the invention, to a limited extent or only in a low content, includes the following substance groups:
(i) straight- or branched-chain saturated paraffins having 20 to 32 C atoms;
(ii) esters having at least 14 C atoms of straight- or branched-chain saturated fatty acids having 8 to 24 C atoms and straight- or branched-chain saturated mono-, di- or triols having 3 to 24 C atoms, these esters containing no free hydroxyl groups;
(iii) esters of benzoic acid and straight- or branched-chain saturated monoalkanols having 10 to 18 C atoms;
(iv) 2,6-naphthalenedicarboxylic acid di-C6-C12 esters;
(v) straight- or branched-chain saturated di-C6-C18-alkyl ethers, in particular (straight-chain) di-C6-C12-alkyl ethers;
(vi) silicone oils from the group consisting of cyclotrisiloxanes, cyclopentasiloxanes, dimethylpolysiloxanes, diethylpolysiloxanes, methylphenylpolysiloxanes, diphenylpolysiloxanes and mixed forms thereof;
(vii) 2-alkyl-1-alkanols having 12 to 32 C atoms of the formula (VII)
   wherein
   Q1 denotes a (preferably straight-chain) alkyl radical having 6 to 18 C atoms and
   Q2 denotes a (preferably straight-chain) alkyl radical having 4 to 16 C atoms.

An oily phase in the narrowest (and most preferred) sense of the present invention. i.e. the substances present, according to the invention, to a limited extent or only in a low content, includes the following substance groups:
(i) straight- or branched-chain saturated paraffins having 20 to 32 C atoms, such as isoeicosane or squalane;
(ii) esters having at least 16 C atoms of straight- or branched-chain saturated fatty acids having 8 to 18 C atoms and straight- or branched-chain saturated mono-, di- or triols having 3 to 18 C atoms, these esters containing no free hydroxyl groups;
(iii) esters of benzoic acid and straight- or branched-chain saturated monoalkanols having 12 to 15 C atoms, specifically C₁₂₋₁₅-alkyl benzoates;
(iv) 2,6-naphthalenedicarboxylic acid di-C6-C10 esters, in particular 2,6-naphthalenedicarboxylic acid di-ethylhexyl ester;
(v) straight-chain di-C6-C10-alkyl ethers, in particular di-n-octyl ether (dicaprylyl ether);
(vi) silicone oils from the group consisting of undecamethylcyclotrisiloxane, cyclomethicone, decamethylcyclopentasiloxane, dimethylpolysiloxanes, diethylpolysiloxanes, methylphenylpolysiloxanes, diphenylpolysiloxanes;
(vii) 2-alkyl-1-alkanols having 12 to 32 C atoms of the formula (VII)
   wherein
   Q1 denotes a (preferably straight-chain) alkyl radical having 6 to 18 C atoms and
   Q2 denotes a (preferably straight-chain) alkyl radical having 4 to 16 C atoms.

Components of type (i) of the oily phase which are to be noted in particular are: isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, 2-ethylhexyl isostearate, isotridecyl isononanoate, 2-ethylhexyl cocoate, caprylic/capric acid triglyceride, and synthetic, semi-synthetic and natural mixtures of such esters, e.g. jojoba oil.

Fatty acid triglycerides (oil components of type (i) of the oily phase) can also be in the form of or as a constituent of synthetic, semi-synthetic and/or natural oils, e.g. olive oil, sunflower oil, soya oil, groundnut oil, rape oil, almond oil, palm oil, coconut oil, palm kernel oil and mixtures thereof.

Oil components of type (vii) of the oily phase which are to be noted in particular are: 2-butyl-1-octanol, 2-hexyl-1-decanol, 2-octyl-1-dodecanol, 2-decyl-tetradecanol, 2-dodecyl-1-hexadecanol and 2-tetradecyl-1-octadecanol.

Oil components which are to be noted in particular are mixtures comprising C₁₂₋₁₅-alkyl benzoate and 2-ethylhexyl isostearate, mixtures comprising C₁₂₋₁₅-alkyl benzoate and isotridecyl isononanoate, mixtures comprising C₁₂₋₁₅-alkyl benzoate, 2-ethylhexyl isostearate and isotridecyl isononanoate, mixtures comprising cyclomethicone and isotridecyl isononanoate and mixtures comprising cyclomethicone and 2-ethylhexyl isostearate.

The formulations according to the invention are preferably a constituent of the following formulations or are in one of the following forms, the maximum weight content according to the invention of the oily phase in the total formulation not being exceeded:

Emulsion of the “oil-in-water” (O/W) type, PIT emulsion, Pickering emulsion, microemulsion, pencil, stick, spray, foam, soaking solution, e.g. for cosmetic wipes, cleansing composition, such as e.g. soap, syndet, liquid washing, shower and bath preparation, skin care composition, cream, lotion, milk, emulsion foam, micro- or nanoemulsion, paste, gel (e.g. hydrogel or hydrodispersion gel), balsam, serum, roll-on, pump spray, aerosol (foaming, non-foaming or after-foaming), foot care composition (including keratolytics, deodorants), insect-repellent composition, sunscreen composition, aftersun preparation, shaving composition, depilatory composition, hair care composition, such as e.g. shampoo, 2-in-1 shampoo, antidandruff shampoo, baby shampoo, shampoo for a dry scalp, shampoo concentrate, conditioner, hair tonic, hair lotion, hair rinse, styling cream, permanent wave and setting composition, hair smoothing composition (straightening composition, relaxer), hair setting composition (spray), styling aid (e.g. gel), as a blonding composition, hair lightener, hair conditioner, hair mousse, hair tint, deodorant and/or antiperspirant; mouthwash and mouth spray, aftershave balm, pre- and aftershave lotion, eye care, make-up, make-up remover, baby article, bath article (e.g. capsule) or mask. It is furthermore advantageous to present the compounds of the formula 1 in encapsulated form, e.g. in liposomes or cellulose capsules.

The cosmetic or dermatological formulations which comprise diphenylmethane derivatives of the formula 1 according to the invention are preferably in the form of an O/W emulsion, the content of the oily phase not exceeding, according to the invention, the values described above.

A formulation according to the invention, in particular in the form of an O/W emulsion, regularly comprises one or more of the following solvents: water or aqueous (salt) solutions, alcohols, diols or polyols of low C number (preferably having 2 to 6 C atoms, specifically having 2 to 4 C atoms), and ethers thereof, preferably ethanol, isopropanol, propylene glycol (1,2-propanediol), glycerol, ethylene glycol, ethylene glycol monoethyl or monobutyl ether, propylene glycol monomethyl, monoethyl or monobutyl ether, diethylene glycol monomethyl or monoethyl ether and analogous products. Mixtures of the above-mentioned solvents are used in particular. In the case of alcoholic solvents, water can be a further constituent.

Further conventional cosmetic auxiliary substances and additives (including water) can be present in amounts of 5-99 wt. %, preferably 10-90 wt. %, based on the total weight of the formulation.

The formulations according to the invention preferably comprise water in an amount in the range of from 25 to 95 wt. %, preferably a water content in the range of from 40 to 90 wt. %, particularly preferably in the range of from 50 to 90 wt. %, in each case based on the total weight of the formulation. The diphenylmethane derivatives of the formula 1 are released from a formulation according to the invention having a water content mentioned (as preferred) and which is preferably in the form of an O/W emulsion better and more effectively with increasing water content.

Those formulations which comprise one or more diphenylmethane derivatives of the formula 1, in particular styrylresorcinol, in a content of from 0.1 to 4 wt. % and in which, as defined above, the oily phase content is 0.1 to 10 wt. % and the water content is 40 to 90 wt. %, in each case based on the total weight of the formulation, are preferred in particular. The contents are chosen such that 100 wt. % is not exceeded.

A formulation according to the invention, in particular in the form of an O/W emulsion, regularly comprises one or more of the following thickeners, which can advantageously be chosen from the group consisting of silicon dioxide, aluminium silicates, polysaccharides or derivatives thereof, e.g. hyaluronic acid, xanthan gum, hydroxypropyl-methylcellulose, particularly advantageously from the group consisting of polyacrylates, preferably a polyacrylate from the group consisting of the so-called Carbopols, for example Carbopols of types 980, 981, 1382, 2984, 5984, in each case individually or in combination.

Formulations according to the invention in the form of an O/W emulsion which comprise diphenylmethane derivatives of the formula 1 advantageously comprise one or more emulsifiers.

O/W emulsifiers are advantageously chosen from the group consisting of polyethoxylated or polypropoxylated or polyethoxylated and polypropoxylated products, e.g.:
the fatty alcohol ethoxylates
the ethoxylated wool wax alcohols,
the polyethylene glycol ethers of the general formula R—O—(—CH₂—CH₂—O—)ₙ—R′,
the fatty acid ethoxylates of the general formula R—COO—(—CH₂—CH₂—O—)ₙ—H,
the etherified fatty acid ethoxylates of the general formula

R—COO—(—CH₂—CH₂—O—)ₙ—R′,
the esterified fatty acid ethoxylates of the general formula

R—COO—(—CH₂—CH₂—O—)ₙ—C(O)—R′,
the polyethylene glycol glycerol fatty acid esters
the ethoxylated sorbitan esters
the cholesterol ethoxylates
the ethoxylated triglycerides
the alkyl ether-carboxylic acids of the general formula

R—COO—(—CH₂—CH₂—O—)ₙ—OOH,wherein n represents a number from 5 to 30,
the polyoxyethylene sorbitol fatty acid esters
the alkyl ether-sulfates of the general formula R—O—(—CH₂—CH₂—O—)ₙ—SO₃—H
the fatty alcohol propoxylates of the general formula R—O—(—CH₂—CH(CH₃)—O—)ₙ—H
the polypropylene glycol ethers of the general formula

R—O—(—CH₂—CH(CH₃)—O—)ₙ—R′
the propoxylated wool wax alcohols,
the etherified fatty acid propoxylates R—COO—(—CH₂—CH(CH₃)—O—)ₙ—R′
the esterified fatty acid propoxylates of the general formula

R—COO—(—CH₂—CH(CH₃)—O—)ₙ—C(O)—R′
the fatty acid propoxylates of the general formula

R—COO—(—CH₂—CH(CH₃)—O—)ₙ—H,
the polypropylene glycol glycerol fatty acid esters
the propoxylated sorbitan esters
the cholesterol propoxylates
the propoxylated triglycerides
the alkyl ether-carboxylic acids of the general formula

R—O—(—CH₂—CH(CH₃)—O—)ₙ—CH₂—COOH,
the alkyl ether-sulfates and the acids on which these sulfates are based of the general formula R—O—(—CH₂—CH(CH₃)—O—)ₙ—SO₃—H,
the fatty alcohol ethoxylates/propoxylates of the general formula R—O—Xₙ—Yₘ—H
the polypropylene glycol ethers of the general formula R—O—Xₙ—Yₘ—R′
the etherified fatty acid propoxylates of the general formula R—COO—Xₙ—Yₘ—R′
the fatty acid ethoxylates/propoxylates of the general formula R—COO—Xₙ—Yₘ—H.

According to the invention, the polyethoxylated or polypropoxylated or polyethoxylated and polypropoxylated O/W emulsifiers employed are particularly advantageously chosen from the group consisting of substances having HLB values of 11-18, very particularly advantageously having HLB values of 14.5-15.5, if the O/W emulsifiers contain saturated radicals R and R′. If the O/W emulsifiers contain unsaturated radicals R and/or R′, or isoalkyl derivatives are present, the preferred HLB value of such emulsifiers can also be lower or higher.

It is of advantage to choose the fatty alcohol ethoxylates from the group consisting of ethoxylated stearyl alcohols, cetyl alcohols and cetylstearyl alcohols (cetearyl alcohols). The following are particularly preferred:

polyethylene glycol (n) stearyl ether (steareth-n), where n=13-20,
polyethylene glycol (n) cetyl ether (ceteth-n), where n=13-20,
polyethylene glycol (n) isocetyl ether (isoceteth-n), where n=13-20,
polyethylene glycol (n) cetylstearyl ether (ceteareth-n), where n=13-20,
polyethylene glycol (m) isostearyl ether (isosteareth-m), where m=12-20
polyethylene glycol (k) oleyl ether (oleth-k), where k=12-15
polyethylene glycol (12) lauryl ether (laureth-12),
polyethylene glycol (12) isolauryl ether (isolaureth-12).

It is furthermore advantageous to chose the fatty acid ethoxylates from the following group:

polyethylene glycol (n) stearate, where n=20-25
polyethylene glycol (m) isostearate, where m=12-25
polyethylene glycol (k) oleate, where k=12-20

Sodium laureth-11 carboxylate can advantageously be used as an ethoxylated alkyl ether-carboxylic acid or salt thereof. Sodium laureth 1-4 sulfate can advantageously be used as an alkyl ether-sulfate. Polyethylene glycol (30) cholesteryl ether can advantageously be used as an ethoxylated cholesterol derivative. Polyethylene glycol (25) soyasterol has also proved suitable.

The polyethylene glycol (60) evening primrose glycerides can advantageously be used as ethoxylated triglycerides.

It is furthermore advantageous to chose the polyethylene glycol glycerol fatty acid esters from the group consisting of

polyethylene glycol (20-23) glyceryl-laurate polyethylene glycol (6) glyceryl-caprylate/caproate, polyethylene glycol (20) glyceryl-oleate, polyethylene glycol (20) glyceryl-isostearate, polyethylene glycol (18) glyceryl-oleate/cocoate.

It is likewise favourable to choose the sorbitan esters from the group consisting of polyethylene glycol (20) sorbitan monolaurate, polyethylene glycol (20) sorbitan monostearate, polyethylene glycol (20) sorbitan monoisostearate, polyethylene glycol (20) sorbitan monopalmitate and polyethylene glycol (20) sorbitan monooleate.

The (in particular topical) cosmetic or therapeutic formulations according to the invention, in particular skin- and hair-lightening compositions, can comprise cosmetic auxiliary substances and additives such as are conventionally used in such formulations, e.g. sunscreen agents, preservatives, bactericides, fungicides, virucides, cooling active compounds, insect repellents (e.g. DEET, IR 3225, Dragorepel), plant extracts, antiinflammatory active compounds, substance which accelerate wound healing (e.g. chitin or chitosan and derivatives thereof), film-forming substances (e.g. polyvinylpyrrolidones or chitosan or derivatives thereof), the usual antioxidants, vitamins (e.g. vitamin C and derivatives, tocopherols and derivatives, vitamin A and derivatives), 2-hydroxycarboxylic acids (e.g. citric acid, malic acid, L-, D- or di-lactic acid), skin care agents (e.g. cholesterol, ceramides, pseuodceramides), softening, moisturizing and/or humectant substances (in particular glycerol, urea or 1,2-alkanediols, such as 1,2-pentanediol, 1,2-hexanediol and/or 1,2-octanediol), saturated fatty acids, mono- or polyunsaturated fatty acids, alpha-hydroxy acids, polyhydroxy-fatty acids or derivatives thereof (e.g. linoleic acid, alpha-linolenic acid, gamma-linolenic acid or arachidonic acid and the particular natural or synthetic esters thereof, waxes or other conventional constituents of a cosmetic or dermatological formulation, such as alcohols, polyols, polymers, foam stabilizers, electrolytes, organic solvents, silicone derivatives or chelating agents (e.g. ethylenediaminetetraacetic acid and derivatives or salts), antidandruff active compounds (e.g. climbazole, ketoconazole, piroctonoleamine, zinc pyrithione), hair care agents, perfume, substances for preventing foaming, dyestuffs, pigments which have a colouring action, thickening agents, surface-active substances, surfactants, emulsifiers, plant parts and plant extracts (e.g. arnica, aloe, beard lichen, ivy, stinging nettle, ginseng, henna, chamomile, marigold, rosemary, sage, blackberry, horsetail or thyme), royal jelly, propolis, proteins, protein hydrolysates, yeast extracts, hop and wheat extracts, peptides or thymus extracts.

The particular amounts of cosmetic or dermatological auxiliary substances and additives and of one or more odoriferous substances (perfumes) to be employed can be easily determined according to the nature of the particular product by simple trials by the person skilled in the art.

The formulations according to the invention which comprise diphenylmethane derivatives of the formula 1 can also comprise further active compounds having a skin-lightening action. According to the invention, all the skin-lightening active compounds which are suitable or usual for cosmetic and/or dermatological uses can be used here. Advantageous skin-lightening active compounds in this respect are kojic acid (5-hydroxy-2-hydroxymethyl-4-pyranone), kojic acid derivatives, such as e.g. kojic acid dipalmitate, arbutin, ascorbic acid, ascorbic acid derivatives, hydroquinone, hydroquinone derivatives, resorcinol, sulfur-containing molecules, such as e.g. glutathione or cysteine, alpha-hydroxy acids (e.g. citric acid, lactic acid, malic acid) and derivatives thereof, N-acetyl-tyrosine and derivatives, undecenoylphenylalanine, gluconic acid, 4-alkylresorcinols, 4-(1-phenylethyl)-1,3-benzenediol, chromone derivatives, such as aloesin, flavonoids, thymol derivatives, 1-aminoethylphosphinic acid, thiourea derivatives, ellagic acid, nicotinamide, zinc salts, such as e.g. zinc chloride or gluconate, thujaplicin and derivatives, triterpenes, such as maslic acid, sterols, such as ergosterol, benzofuranones, such as senkyunolide, vinyl- and ethylguaiacol, dionic acids, such as octadecenedionic acid and azelaic acid, inhibitors of nitrogen oxide synthesis, such as e.g. L-nitroarginine and derivatives thereof, 2,7-dinitroindazole or thiocitrulline, metal chelators (e.g. alpha-hydroxy-fatty acids, palmitic acid, phytic acid, lactoferrin, humic acid, bile acid, bile extracts, bilirubin, biliverdin, EDTA, EGTA and derivatives thereof, retinoids, soya milk and extract, serine protease inhibitors or liponic acid or other synthetic or natural active compounds for lightening of the skin and hair, the latter also being used in the form of an extract from plants, such as e.g. bearberry extract, rice extract, papaya extract, liquorice root extract or constituents concentrated therefrom, such as glabridin or licochalcone A, Artocarpus extract, extract from Rumex and Ramulus species, extracts from pine species (Pinus) and extracts from Vitis species or stilbene derivatives concentrated therefrom, extract from Saxifraga, mulberry, Scutelleria or/and grape.

For use, the cosmetically and/or dermatologically active formulations according to the invention comprising diphenylmethane derivatives of the formula 1 are applied to the skin and/or hair in a sufficient amount in the conventional manner for cosmetics and dermatics. In this context, those cosmetic and dermatological formulations which additionally comprise one or more sunscreen filters (UV absorbers, UV filters) and thereby act both as hair- or skin-lightening or senile keratosis-reducing compositions and as sunscreen compositions offer particular advantages.

The diphenylmethane derivatives of the formula 1 according to the invention or to be employed according to the invention are particularly preferably combined with water-soluble UV filters, in a preferred embodiment with phenylene-bis-benzimidazyl-tetrasulfonic acid disodium salt (Neo Heliopan® AP).

Such combinations are particularly advantageous if the formulation according to the invention having a low oil content has a sunscreen factor of greater than or equal to 8 (preferably greater than or equal to 15). In this context, such a formulation furthermore preferably has a pH of less than or equal to 6, more preferably of less than or equal to 5.5.

Surprisingly, it has now been found that UV filters can improve the stability of the diphenylmethane derivatives of the formula 1 in formulations according to the invention. In particular, UV filters can prevent or slow down a discolouration of the diphenylmethane derivatives of the formula 1 caused by sunlight or other light. Both are important in particular in cosmetic formulations. According to the invention, UV filters are therefore used to stabilize the diphenylmethane derivatives of the formula 1, in particular by employing one or more UV filters in a formulation according to the invention in an amount sufficient to stabilize the diphenylmethane derivatives of the formula 1.

Formulations according to the invention comprising one or more sunscreen filters (UV absorbers) preferably have a total content of UV absorbers in the range of from 0.1 to 30 wt. %, preferably in the range of from 0.2 to 20 wt. %, in particular 0.5 to 15 wt. %, based on the total weight of the formulation.

In a preferred embodiment, a formulation according to the invention comprises a total amount of UV filters which is capable of effecting stabilization of the diphenylmethane derivatives of the formula 1 in a formulation according to the invention and therefore of preventing a discolouration of the formulation according to the invention. For the purposes of stabilization, the total amount of UV filters is preferably in the range of from 0.1 to 2 wt. %, in particular 0.2 to 1 wt. %, based on the total weight of the formulation.

In a further preferred embodiment, a formulation according to the invention comprises a total amount of UV filters and/or inorganic pigments such that the formulation according to the invention has a sunscreen factor of greater than or equal to 2 (preferably greater than or equal to 5). These sunscreen compositions are suitable for protecting the skin and hair.

In this context, formulations according to the invention additionally comprising one or more sunscreen filters (UV absorbers) can be in various forms such as are conventionally employed e.g. for sunscreen formulations. Thus, they can be e.g. in the form of an emulsion of the oil-in-water (O/W) type, a gel, a hydrodispersion, or also an aerosol.

The formulations according to the invention advantageously comprise at least one UV-A filter and/or at least one UV-B filter and/or a broadband filter and/or at least one inorganic pigment. Formulations according to the invention preferably comprise at least one UV-B filter or one broadband filter, and furthermore preferably at least one UV-A filter and at least one UV-B filter.

Suitable sunscreen agents (UV absorbers) are e.g. organic UV absorbers from the class consisting of 4-aminobenzoic acid and derivatives, salicylic acid derivatives, benzophenone derivatives, dibenzoylmethane derivatives, diphenyl acrylates, 3-imidazol-4-yl-acrylic acid and esters thereof, benzofuran derivatives, benzylidenemalonate derivatives, polymeric UV absorbers, containing one or more organosilicon radicals, cinnamic acid derivatives, camphor derivatives, trianilino-s-triazine derivatives, 2-hydroxyphenylbenzotriazole derivatives, phenylbenzimidazolesulfonic acid derivatives and salts thereof, anthranilic acid menthyl ester, benzotriazole derivatives, indole derivatives.

The UV absorbers mentioned below, which can be employed in the context of the present invention, are preferred, but of course not limiting.

Advantageous UV filters are

UV-B filters, such as e.g.:
p-aminobenzoic acid
p-aminobenzoic acid ethyl ester (25 mol) ethoxylated
p-dimethylaminobenzoic acid 2-ethylhexyl ester
p-aminobenzoic acid ethyl ester (2 mol) N-propoxylated
p-aminobenzoic acid glycerol ester
salicylic acid homomethyl ester (homosalate) (Neo-Heliopan®HMS)
salicylic acid 2-ethylhexyl ester (Neo-Heliopan®OS)
triethanolamine salicylate
4-isopropylbenzyl salicylate
anthranilic acid menthyl ester (Neo Heliopan®MA)
diisopropylcinnamic acid ethyl ester
p-Methoxycinnamic acid 2-ethylhexyl ester (Neo Heliopan®AV)
diisopropylcinnamic acid methyl ester
p-methoxycinnamic acid isoamyl ester (Neo Heliopan®E 1000)
p-methoxycinnamic acid diethanolamine salt
p-methoxycinnamic acid isopropyl ester
2-phenylbenzimidazolesulfonic acid and salts (Neo Heliopan®Hydro)
3-(4′-trimethylammonium)-benzylidene-bornan-2-one methyl sulfate
β-imidazole-4(5)-acrylic acid (urocanic acid)
3-(4′-sulfo)benzylidene-bornan-2-one and salts
3-(4′-methylbenzylidene)-d,l-camphor (Neo Heliopan®MBC)
3-benzylidene-d,l-camphor
N-[(2 and 4)-[2-(oxoborn-3-ylidene)methyl]benzyl]-acrylamide polymer
4,4′-[(6-[4-(1,1-dimethyl)-aminocarbonyl)-phenylamino]-1,3,5-triazine-2,4-diyl)diimino]-bis-(benzoic acid 2-ethylhexyl ester) (Uvasorb®HEB)
benzylidenemalonate-polysiloxane (Parsol®SLX)
glyceryl ethylhexanoate dimethoxycinnamate
dipropylene glycol salicylate
tris(2-ethylhexyl) 4,4′,4″-(1,3,5-triazine-2,4,6-triyltriimino)tribenzoate (Uvinul®T150)
   broadband filters, such as e.g.:
2-ethylhexyl 2-cyano-3,3-diphenylacrylate (Neo Heliopan®303)
ethyl 2-cyano-3,3′-diphenylacrylate
2-hydroxy-4-methoxybenzophenone (Neo Heliopan®BB)
2-hydroxy-4-methoxybenzophenone-5-sulfonic acid
dihydroxy-4-methoxybenzophenone
2,4-dihydroxybenzophenone
tetrahydroxybenzophenone
2,2′-dihydroxy-4,4′-dimethoxybenzophenone
2-hydroxy-4-n-octoxybenzophenone
2-hydroxy-4-methoxy-4′-methylbenzophenone
sodium hydroxymethoxybenzophenone sulfonate
disodium 2,2′-dihydroxy-4,4′-dimethoxy-5,5′-disulfo-benzophenone
phenol, -(2H-benzotriazol-2-yl)-4-methyl-6-(2-methyl-3-(1,3,3,3-tetramethyl-1-(trimethylsilyl)-oxy)-disiloxyanyl)-propyl), (Mexoryl®XL)
2,2′-methylene-bis-(6-(2H-benztriazol-2-yl)-4-(1,1,3,3-tetramethylbutyl)-phenol), (Tinosorb®M)
2,4-bis-[4-(2-ethylhexyloxy)-2-hydroxyphenyl]-1,3,5-triazine
2,4-bis-[{(4-(2-ethyl-hexyloxy)-2-hydroxy}-phenyl]-6-(4-methoxyphenyl)-1,3,5-triazine, (Tinosorb®S)
2,4-bis-[{(4-(3-sulfonato)-2-hydroxy-propyloxy)-2-hydroxy}-phenyl]-6-(4-methoxyphenyl)-1,3,5-triazine sodium salt
2,4-bis-[{(3-(2-propyloxy)-2-hydroxy-propyloxy)-2-hydroxy}-phenyl]-6-(4-methoxy-phenyl)-1,3,5-triazine
2,4-bis-[{4-(2-ethyl-hexyloxy)-2-hydroxy}-phenyl]-6-[4-(2-methoxyethyl-carbonyl)-phenylamino]-1,3,5-triazine
2,4-bis-[{(3-(2-propyloxy)-2-hydroxy-propyloxy)-2-hydroxyl}-phenyl]-6-[4-(2-ethylcarboxyl)-phenylamino]-1,3,5-triazine
2,4-bis-[{(2-ethyl-hexyloxy)-2-hydroxy}-phenyl]-6-(1-methyl-pyrrol-2-yl-)-1,3,5-triazine
2,4-bis-[{4-tris-(trimethylsiloxy-silylpropyloxy)-2-hydroxy}-phenyl]-6-(4-methoxyphenyl)-1,3,5-triazine
2,4-bis-[{4-(2″-methylpropenyloxy)-2-hydroxy}-phenyl]-6-(4-methoxyphenyl)-1,3,5-triazine
2,4-bis-[{4-(1′,1′,1′,3′5′,5′,5′-Heptamethylsiloxy-2″-methyl-propyloxy)-2-hydroxy}-phenyl]-6-(4-methoxyphenyl)-1,3,5-triazine
   UV-A filters, such as e.g.:
Terephthalylidene-dibornanesulfonic acid and salts (Mexoryl®SX)
4-t-Butyl-4′-methoxy-dibenzoylmethane (avobenzone)/(Neo Heliopan®357)
phenylene-bis-benzimidazyl-tetrasulfonic acid disodium salt (Neo Heliopan®AP)
2,2′-(1,4-phenylene)-bis-(1H-benzimidazole-4,6-disulfonic acid), monosodium salt
2-(4-diethylamino-2-hydroxybenzoyl)-benzoic acid hexyl ester (Uvinul® A Plus)
4-isopropyldibenzoylmethane
Indanylidene compounds according to DE 100 55 940 (=WO 02/38537)

In this context, UV absorbers which are particularly suitable for combination are
p-aminobenzoic acid
3-(4′-trimethylammonium)-benzylidene-bornan-2-one methyl sulfate
salicylic acid homomethyl ester (Neo-Heliopan®HMS)
2-hydroxy-4-methoxy-benzophenone (Neo Heliopan®BB)
2-phenylbenzimidazolesulfonic acid (Neo Heliopan®Hydro)
terephthalylidene-dibornanesulfonic acid and salts (Mexoryl®SX)
4-tert-butyl-4′-methoxydibenzoylmethane (Neo Heliopan®357)
3-(4′-sulfo)benzylidene-bornan-2-one and salts
2-ethylhexyl 2-cyano-3,3-diphenylacrylate (Neo Heliopan®303)
N-[(2 and 4)-[2-(oxoborn-3-ylidene)methyl]benzyl]-acrylamide polymer
p-methoxycinnamic acid 2-ethylhexyl ester (Neo Heliopan® AV)
p-aminobenzoic acid ethyl ester (25 mol) ethoxylated
p-methoxycinnamic acid isoamyl ester (Neo Heliopan®E 1000)
2,4,6-trianilino-(p-carbo-2′-ethylhexyl-1′-oxy)-1,3,5-triazine (Uvinal®T150)
phenol, 2-(2H-benzotriazol-2-yl)-4-methyl-6-(2-methyl-3-(1,3,3,3-tetramethyl-1-(trimethylsilyl)-oxy)-disiloxyanyl)-propyl), (Mexoryl®XL)
4,4′-[(6-[4-(1,1-dimethyl)-aminocarbonyl)-phenylamino]-1,3,5-triazine-2,4-diyl)-diimino]-bis-(benzoic acid 2-ethylhexyl ester), (UvasorbHEB)
3-(4′-methylbenzylidene)-d,l-camphor (Neo Heliopan®MBC)
3-benzylidenecamphor
salicylic acid 2-ethylhexyl ester (Neo-Heliopan®OS)
4-dimethylaminobenzoic acid 2-ethylhexyl ester (Padimate O)
hydroxy-4-methoxy-benzophenone-5-sulfonic acid and Na salt
2,2′-methylene-bis-(6-(2H-benzotriazol-2-yl)-4-1,1,3,3-tetramethylbutyl)-phenol), (Tinosorb®M)
phenylene-bis-benzimidazyl-tetrasulfonic acid disodium salt (Neo Heliopan®AP)
2,4-bis-[{(4-(2-ethyl-hexyloxy)-2-hydroxy}-phenyl]-6-(4-methoxyphenyl)-1,3,5-triazine, (Tinosorb®S)
benzylidenemalonate-polysiloxane (Parsol®SLX)
menthyl anthranilate (Neo Heliopan®MA)
2-(4-diethylamino-2-hydroxybenzoyl)-benzoic acid hexyl ester (Uvinul® A Plus)
indanylidene compounds according to DE 100 55 940 (=WO 02/38537)

Particulate UV filters or inorganic pigments, which can optionally be hydrophobized, such as the oxides of titanium (TiO₂), zinc (ZnO), iron (Fe₂O₃), zirconium (ZrO₂), silicon (SiO₂), manganese (e.g. MnO), aluminium (Al₂O₃), cerium (e.g. Ce₂O₃) and/or mixtures, can furthermore be employed.

Formulations according to the invention regularly comprise a content of (skin and/or hair) care substances in the range of from 0.01 to 10 wt. %, preferably in the range of from 0.1 to 8 wt. %. According to a preferred embodiment, the compositions comprise one or more animal and/or plant fats and oils (which are then a constituent of the oily phase) having care properties, such as olive oil, sunflower oil, refined soya oil, palm oil, sesame oil, rape oil, almond oil, borage oil, evening primrose oil, coconut oil, shea butter, jojoba oil, sperm oil, beef tallow, neat's foot oil and lard.

Formulations according to the invention optionally comprise further constituents having care properties, such as, for example, fatty alcohols having 6-30 C atoms. The fatty alcohols here can be saturated or unsaturated and linear or branched. Furthermore, these fatty alcohols can in some cases be a constituent of the oily phase (vii) if they correspond to the definition given there. Alcohols which can be employed are, for example, decanol, decenol, octanol, octenol, dodecanol, dodecenol, octadienol, decadienol, dodecadienol, oleyl alcohol, ricinoleyl alcohol, erucyl alcohol, stearyl alcohol, isostearyl alcohol, cetyl alcohol, lauryl alcohol, myristyl alcohol, arachidyl alcohol, caprylyl alcohol, capryl alcohol, linoleyl alcohol, linolenyl alcohol and behenyl alcohol, as well as Guerbet alcohols thereof, such as, for example, 2-octyl-1-dodecanol, it being possible for the list to be extended virtually as desired by further alcohols of related structural chemistry. The fatty alcohols preferably originate from natural fatty acids, being conventionally prepared from the corresponding esters of the fatty acids by reduction. Fatty alcohol fractions which are formed by reduction from naturally occurring fats and fatty oils, such as e.g. beef tallow, groundnut oil, colza oil, cottonseed oil, soya oil, sunflower oil, palm kernel oil, linseed oil, maize oil, castor oil, rape oil, sesame oil, cacao butter and coconut fat, can furthermore be employed.

Substances having care properties which can be employed in an outstanding manner in the formulations according to the invention comprising diphenylmethane derivatives of the formula 1 moreover include
ceramides, where ceramides are understood as meaning N-acylsphingosins (fatty acid amides of sphingosin) or synthetic analogues of such lipids (so-called pseudo-ceramides), which significantly improve the water retention capacity of the stratum corneum.
phospholipids, for example soya lecithin, egg lecithin and cephalins
fatty acids
phytosterols and phytosterol-containing fats or waxes
vaseline, paraffin oils and silicone oils; the latter include, inter alia, dialkyl- and alkylarylsiloxanes, such as dimethylpolysiloxane and methylphenylpolysiloxane, as well as alkoxylated and quaternized derivatives thereof.

Animal and/or plant protein hydrolysates can advantageously also be added to the formulations according to the invention. Substances which are advantageous in this respect are, in particular, elastin, collagen, keratin, milk protein, soya protein, oat protein, pea protein, almond protein and wheat protein fractions or corresponding protein hydrolysates, and also condensation products thereof with fatty acids and quaternized protein hydrolysates, the use of plant protein hydrolysates being preferred.

The formulations according to the invention which comprise diphenylmethane derivatives of the formula 1 can also comprise antioxidants, it being possible for all the antioxidants which are suitable or usual for cosmetic and/or dermatological uses to be used. The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. alpha-carotene, beta-carotene, lycopene) and derivatives thereof, liponic acid and derivatives thereof (e.g. dihydroliponic acid), aurothioglucose, propyl-thiouracil and other thiols (e.g. thioredoxin, glutathione, cysteine, cystine, cystamine and glycosyl, N-acetyl, methyl, ethyl, propyl, amyl, butyl and lauryl, palmitoyl, oleyl, gamma-linoleyl, cholesteryl and glyceryl esters thereof) as well as salts thereof, dilauryl thiodipropionate, distearyl thiodipropionate, thiodipropionic acid and derivatives thereof (esters, ethers, peptides, lipids, nucleotides, nucleosides and salts) as well as sulfoximine compounds (e.g. buthionine sulfoximine, homocysteine sulfoximine, buthionine sulfones, penta-, hexa-, heptathionine sulfoximine) in very low tolerated dosages, furthermore (metal) chelators, e.g. alpha-hydroxy-fatty acids, palmitic acid, phytic acid, lactoferrin, alpha-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives thereof (e.g. vitamin E, vitamin E acetate), vitamin A and derivatives thereof (vitamin A palmitate) as well as coniferylbenzoate of benzoin resin, rutic acid and derivatives thereof, ferulic acid and derivatives thereof, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenium methionine), stilbenes and derivatives thereof (e.g. stilbene oxide, trans-stilbene oxide) and derivatives (salts, esters, ethers, sugars, nucleotides, nucleosides, peptides and lipids) of these active compounds mentioned.

The formulations according to the invention which comprise diphenylmethane derivatives of the formula 1 can advantageously also comprise vitamins and vitamin precursors, it being possible for all the vitamins and vitamin precursors which are suitable or usual for cosmetic and/or dermatological uses to be used.

There are worth mentioning here, in particular, vitamins and vitamin precursors, such as tocopherols, vitamin A, niacin acid and niacinamide, further vitamins of the B complex, in particular biotin, and vitamin C and panthenol and derivatives thereof, in particular the esters and ethers of panthenol and cationically derivatized panthenols, such as e.g. panthenol triacetate, panthenol monoethyl ether and the monoacetate thereof and cationic panthenol derivatives.

The formulations according to the invention, which advantageously comprise diphenylmethane derivatives of the formula 1, can also comprise antiinflammatory and/or redness- and/or itching-alleviating active compounds. All the antiinflammatory or redness- and/or itching-alleviating active compounds which are suitable or usual for cosmetic and/or dermatological uses can be used here. Antiinflammatory and redness- and/or itching-alleviating active compounds which are advantageously employed are steroidal antiinflammatory substances of the corticosteroid type, such as e.g. hydrocortisone, dexamethasone, dexamethasone phosphate, methylprednisolone or cortisone, it being possible for the list to be extended by addition of further steroidal antiinflammatories. Non-steroidal antiinflammatories can also be employed. There are to be mentioned here by way of example oxicams, such as piroxicam or tenoxicam; salicylates, such as aspirin, Disalcid, Solprin or fendosal; acetic acid derivatives, such as diclofenac, fenclofenac, indomethacin, sulindac, tolmetin or clindanac; fenamates, such as mefenamic, meclofenamic, flufenamic or niflumic; propionic acid derivatives, such as ibuprofen, naproxen, benoxaprofen or pyrazoles, such as phenylbutazone, oxyphenylbutazone, febrazone or azapropazone. Alternatively, natural antiinflammatory or redness- and/or itching-alleviating substances can be employed. Plant extracts, specific highly active plant extract fractions and highly pure active substances isolated from plant extracts can be employed. Extracts, fractions and active substances from chamomile, aloe vera, Commiphora species, Rubia species, willow, rose-bay willow herb, oats as well as pure substances, such as, inter alia, bisabolol, apigenin 7-glucoside, boswellic acid, phytosterols, glycyrrhizic acid, glabridin or licochalcone A, are particularly preferred. The formulations comprising diphenylmethane derivatives of the formula 1 can also comprise mixtures of two or more antiinflammatory active compounds.

Bisabolol, boswellic acid, as well as extracts and isolated highly pure active compounds from oats and Echinacea are particularly preferred for use in the context of the invention, and alpha-bisabolol and extracts and isolated highly pure active compounds from oats are especially preferred.

The amount of antiirritants (one or more compounds) in the formulations is preferably 0.0001 to 20 wt. %, particularly preferably 0.0001 to 10 wt. %, in particular 0.001 to 5 wt. %, based on the total weight of the formulation.

The formulations according to the invention which comprise diphenylmethane derivatives of the formula 1 can advantageously also comprise moisture retention regulators. The following substances e.g. are used as moisture retention regulators (moisturizers): sodium lactate, urea, alcohols, sorbitol, glycerol, propylene glycol, collagen, elastin or hyaluronic acid, diacyl adipates, petrolatum, ectoin, urocanic acid, lecithin, pantheol, phytantriol, lycopene, algae extract, ceramides, cholesterol, glycolipids, chitosan, chondroitin sulfate, polyamino acids, lanolin, lanolin esters, amino acids, alpha-hydroxy acids (e.g. citric acid, lactic acid, malic acid) and derivatives thereof, sugars (e.g. inositol), alpha-hydroxy-fatty acids, phytosterols, triterpene acids, such as betulinic acid or ursolic acid, algae extracts.

The formulations according to the invention which comprise diphenylmethane derivatives of the formula 1 can advantageously also comprise mono-, di- and oligosaccharides, such as, for example, glucose, galactose, fructose, mannose, laevulose and lactose.

The formulations according to the invention which comprise diphenylmethane derivatives of the formula 1 can advantageously also comprise plant extracts, which are conventionally prepared by extraction of the whole plant, but also in individual cases exclusively from blossom and/or leaves, wood, bark or roots of the plant. In respect of the plant extracts which can be used, reference is made in particular to the extracts which are listed in the table starting on page 44 of the 3rd edition of the Leitfaden zur Inhaltsstoffdeklaration kosmetischer Mittel [Manual of Declaration of the Constituents of Cosmetic Compositions], published by Industrieverband Körperpflegemittel und Waschmittel e.V. (IKW), Frankfurt. Extracts which are advantageous in particular are those from aloe, witch hazel, algae, oak bark, rose-bay willow-herb, stinging nettle, dead nettle, hops, chamomile, yarrow, arnica, calendula, burdock root, horsetail, hawthorn, linden blossom, almond, pine needle, horse chestnut, sandalwood, juniper, coconut, mango, apricot, orange, lemon, lime, grapefruit, apple, green tea, grapefruit pip, wheat, oats, barley, sage, thyme, wild thyme, rosemary, birch, mallow, lady's smock, willow bark, restharrow, coltsfoot, hibiscus, ginseng and ginger root. In this context, the extracts from aloe vera, chamomile, algae, rosemary, calendula, ginseng, cucumber, sage, stinging nettle, linden blossom, arnica and witch hazel are particularly preferred. Mixtures of two or more plant extracts can also be employed. Extraction agents which can be used for the preparation of the plant extracts mentioned are, inter alia, water, alcohols and mixtures thereof. In this context, among the alcohols lower alcohols, such as ethanol and isopropanol, but also polyhydric alcohols, such as ethylene glycol, propylene glycol and butylene glycol, are preferred, and in particular both as the sole extraction agent and in mixtures with water. The plant extracts can be employed both in the pure and in the diluted form.

Formulations according to the invention can in numerous cases advantageously comprise the following preservatives. Preservatives which are preferably chosen here are those such as benzoic acid, its esters and salts, propionic acid and its salts, salicylic acid and its salts, 2,4-hexadienoic acid (sorbic acid) and its salts, formaldehyde and paraformaldehyde, 2-hydroxybiphenyl ether and its salts, 2-zinc-sulfidopyridine N-oxide, inorganic sulfites and bisulfites, sodium iodate, chlorobutanolum, 4-ethylmercury(II)-5-amino-1,3-bis(2-hydroxybenzoic acid), its salts and esters, dehydracetic acid, formic acid, 1,6-bis(4-amidino-2-bromophenoxy)-n-hexane and its salts, the sodium salt of ethylmercury(II)-thiosalicylic acid, phenylmercury and its salts, 10-undecylenic acid and its salts, 5-amino-1,3-bis(2-ethylhexyl)-5-methyl-hexahydropyrimidine, 5-bromo-5-nitro-1,3-dioxane, 2-bromo-2-nitro-1,3-propanediol, 2,4-dichlorobenzyl alcohol, N-(4-chlorophenyl)-N′-(3,4-dichlorophenyl)-urea, 4-chloro-m-cresol, 2,4,4′-trichloro-2′-hydroxy-diphenyl ether, 4-chloro-3,5-dimethylphenol, 1,1′-methylene-bis(3-(1-hydroxymethyl-2,4-dioximidazolidin-5-yl)urea), poly-(hexamethylene diguanide) hydrochloride, 2-phenoxyethanol, hexamethylenetetramine, 1-(3-chloroallyl)-3,5,7-triaza-1-azonia-adamantane chloride, 1-(4-chlorophenoxy)-1-(1H-imidazol-1-yl)-3,3-dimethyl-2-butanone, 1,3-bis-(hydroxy-methyl)-5,5-dimethyl-2,4-imidazolidinedione, benzyl alcohol, Octopirox, 1,2-dibromo-2,4-dicyanobutane, 2,2′-methylene-bis(6-bromo-4-chloro-phenol), bromochlorophene, mixture of 5-chloro-2-methyl-3(2H)-isothiazolinone and 2-methyl-3(2H)-isothiazolinone with magnesium chloride and magnesium nitrate, 2-benzyl-4-chlorophenol, 2-chloroacetamide, chlorhexidine, chlorhexidine acetate, chlorhexidine gluconate, chlorhexidine hydrochloride, 1-phenoxy-propan-2-ol, N-alkyl(C₁₂-C₂₂)trimethyl-ammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxy-methylurea, 1,6-bis(4-amidino-phenoxy)-n-hexane and its salts, glutaraldehyde, 5-ethyl-1-aza-3,7-dioxabicyclo(3.3.0)octane, 3-(4-chlorophenoxy)-1,2-propanediol, hyamines, alkyl-(C₈-C₁₈)-dimethyl-benzyl-ammonium chloride, alkyl-(C₈-C₁₈)-dimethyl-benzylammonium bromide, alkyl-(C₈-C₁₈)-dimethyl-benzyl-ammonium saccharinate, benzyl hemiformal, 3-iodo-2-propynyl butylcarbamate or sodium hydroxymethyl-aminoacetate.

In various cases it may also be advantageous also to employ substances which are chiefly employed for inhibition of the growth of undesirable microorganisms on or in animal organisms in the formulations according to the invention comprising diphenylmethane derivatives of the formula 1. In this respect, in addition to conventional preservatives, further active compounds which are worth mentioning, in addition to the large group of conventional antibiotics, are, in particular, the products relevant for cosmetics, such as triclosan, climbazole, octoxyglycerol, Octopirox (1-hydroxy-4-methyl-6-(2,4,4-trimethylpentyl)-2(1H)-pyridone, 2-aminoethanol), chitosan, farnesol, glycerol monolaurate or combinations of the substances mentioned, which are employed, inter alia, against underarm odour, foot odour or dandruff formation.

Substances having a perspiration-inhibiting activity (antiperspirants) can moreover be particularly advantageously employed in the formulations according to the invention comprising diphenylmethane derivatives of the formula 1, for combating body odour. Perspiration-inhibiting active compounds which are employed are, above all, aluminium salts, such as aluminium chloride, aluminium hydrochloride, nitrate, sulfate, acetate etc. In addition, however, the use of compounds of zinc, magnesium and zirconium may also be advantageous. For use in cosmetic and dermatological antiperspirants, the aluminium salts and—to a somewhat lesser extent—aluminium/zirconium salt combinations have substantially proved suitable. The aluminium hydroxychlorides which are partly neutralized and therefore tolerated better by the skin, but are not quite so active, are additionally worth mentioning. Alongside aluminium salts, further substances are also possible, such as, for example, a) protein-precipitating substances, such as, inter alia, formaldehyde, glutaraldehyde, natural and synthetic tannins and trichloroacetic acid, which bring about surface blockage of the sweat glands, b) local anaesthetics (inter alia dilute solutions of e.g. lidocaine, prilocaine or mixtures of such substances), which eliminate sympathetic supply of the sweat glands by blockade of the peripheral nerve pathways, c) zeolites of the X, A or Y type, which, alongside the reduction in secretion of perspiration, also function as adsorbents for bad odours, and d) botulinus toxin (toxin of the bacterium Clostridium botulinum), which is also employed in cases of hyperhidrosis, a pathologically increased secretion of perspiration, and the action of which is based on an irreversible blocking of the release of the transmitter substance acetylcholine, which is relevant for secretion of perspiration.

Individual cooling active compounds which are preferred for use in the context of the present invention are listed below. The person skilled in the art can supplement the following list with a large number of further cooling active compounds; the cooling active compounds listed can also be employed in combination with one another: l-menthol, d-menthol, racemic menthol, menthone glycerol acetal (trade name: Frescolat®MGA), menthyl lactate (trade name: Frescolat®ML, menthyl lactate is preferably l-menthyl lactate, in particular l-menthyl l-lactate), substituted menthyl-3-carboxylic acid amides (e.g. menthyl-3-carboxylic acid N-ethylamide), 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, N-acetylglycine menthyl ester, isopulegol, menthyl hydroxycarboxylic acid esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl 3,6-di- and -trioxaalkanoates, 3-menthyl methoxyacetate, icilin.

Preferred cooling active compounds are: l-menthol, d-menthol, racemic menthol, menthone glycerol acetal (trade name: Frescolat®MGA), menthyl lactate (preferably l-menthyl lactate, in particular l-menthyl l-lactate, trade name: Frescolat®ML), substituted menthyl-3-carboxylic acid amides (e.g. menthyl-3-carboxylic acid N-ethylamide), 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, isopulegol.

Particularly preferred cooling active compounds are: l-menthol, racemic menthol, menthone glycerol acetal (trade name: Frescolat®MGA), menthyl lactate (preferably l-menthyl lactate, in particular l-menthyl l-lactate, trade name: Frescolat®ML), 3-menthoxypropane-1,2-diol, 2-hydroxyethyl menthyl carbonate, 2-hydroxypropyl menthyl carbonate.

Very particularly preferred cooling active compounds are: l-menthol, menthone glycerol acetal (trade name: Frescolat®MGA), menthyl lactate (preferably l-menthyl lactate, in particular l-menthyl l-lactate, trade name: Frescolat®ML).

The use concentration of the cooling active compounds to be employed is, depending on the substance, preferably in the concentration range of from 0.01 to 20 wt. % and preferably in the concentration range of from 0.1 to 5 wt. %, based on the total weight of the finished (ready-to-use) cosmetic or pharmaceutical formulation.

The formulations according to the invention which comprise diphenylmethane derivatives of the formula 1 can also comprise anionic, cationic, nonionic and/or amphoteric surfactants, especially if crystalline or microcrystalline solids, for example inorganic micropigments, are to be incorporated into the formulations. Surfactants are amphiphilic substances which can dissolve organic, nonpolar substances in water. According to the invention, surfactants therefore do not belong to the oily phase. In this context, the hydrophilic contents of a surfactant molecule are usually polar functional groups, for example —COO⁻, —OSO₃²⁻, —SO₃⁻, while the hydrophobic parts as a rule are nonpolar hydrocarbon radicals. Surfactants are in general classified according to the nature and charge of the hydrophilic molecular moiety. A distinction can be made between four groups here:
anionic surfactants,
cationic surfactants,
amphoteric surfactants and
nonionic surfactants.

Anionic surfactants as a rule contain carboxylate, sulfate or sulfonate groups as functional groups. In aqueous solution, they form negatively charged organic ions in an acid or neutral medium. Cationic surfactants are almost exclusively characterized by the presence of a quaternary ammonium group. In aqueous solution, they form positively charged organic ions in an acid or neutral medium. Amphoteric surfactants contain both anionic and cationic groups and accordingly behave like anionic or cationic surfactants in aqueous solution, depending on the pH. In a strongly acid medium they have a positive charge, and in an alkaline medium a negative charge. On the other hand, they are zwitter-ionic in the neutral pH range. Polyether chains are typical of nonionic surfactants. Nonionic surfactants do not form ions in an aqueous medium.

### A. Anionic Surfactants

Anionic surfactants which are advantageously to be used are acylamino acids (and salts thereof), such as
acyl glutamates, for example sodium acyl glutamate, di-TEA-palmitoyl aspartate and sodium caprylic/capric glutamate,
acyl peptides, for example palmitoyl hydrolysed milk protein, sodium cocoyl hydrolysed soya protein and sodium/potassium cocoyl hydrolysed collagen,
sarcosinates, for example myristoyl sarcosine, TEA-lauroyl sarcosinate, sodium lauroyl sarcosinate and sodium cocoyl sarcosinate,
taurates, for example sodium lauroyl taurate and sodium methylcocoyl taurate,
acyl lactylates, lauroyl lactylate, caproyl lactylate
alaninates
   carboxylic acids and derivatives, such as
for example, lauric acid, aluminium stearate, magnesium alkanolate and zinc undecylenate,
ester-carboxylic acids, for example calcium stearoyl lactylate, laureth-6 citrate and sodium PEG-4 lauramide carboxylate,
ether-carboxylic acids, for example sodium laureth-13 carboxylate and sodium PEG-6 cocamide carboxylate,
   phosphoric acid esters and salts, such as, for example, DEA-oleth-10 phosphate and dilaureth-4 phosphate,
   sulfonic acids and salts, such as
acyl isethionates, e.g. sodium/ammonium cocoyl isethionate,
alkylarylsulfonates,
alkylsulfonates, for example sodium coco-monoglyceride sulfate, sodium C₁₂₋₁₄ olefin-sulfonate, sodium lauryl sulfoacetate and magnesium PEG-3 cocamide sulfate,
sulfosuccinates, for example dioctyl sodium sulfosuccinate, disodium laureth-sulfosuccinate, disodium laurylsulfosuccinate and disodium undecylenamido-MEA-sulfosuccinate
   and
   sulfuric acid esters, such as
alkyl ether-sulfate, for example sodium, ammonium, magnesium, MIPA, TIPA laureth sulfate, sodium myreth sulfate and sodium C12-13 pareth sulfate,
alkyl sulfates, for example sodium, ammonium and TEA lauryl sulfate.

### B. Cationic Surfactants

Cationic surfactants which are advantageously to be used are
alkylamines,
alkylimidazoles,
ethoxylated amines and
quaternary surfactants,
   RNH₂CH₂CH₂COO⁻ (at pH=7)
   RNHCH₂CH₂COO—B⁺ (at pH=12) B⁺=any desired cation, e.g. Na⁺
ester quats

Quaternary surfactants contain at least one N atom which is covalently bonded to 4 alkyl or aryl groups. This leads to a positive charge, independently of the pH. Alkylbetaine, alkylamidopropylbetaine and alkylamidopropylhydroxysulfaine are advantageous. The cationic surfactants used can furthermore preferably be chosen from the group consisting of quaternary ammonium compounds, in particular benzyltrialkyl-ammonium chlorides or bromides, such as, for example, benzyldimethylstearyl-ammonium chloride, furthermore alkyltrialkylammonium salts, for example cetyltrimethylammonium chloride or bromide, alkyldimethylhydroxy-ethylammonium chlorides or bromides, dialkyldimethylammonium chlorides or bromides, alkylamide-ethyltrimethyl-ammonium ether-sulfates, alkylpyridinium salts, for example lauryl- or cetylpyrimidinium chloride, imidazoline derivatives and compounds having a cationic character, such as amine oxides, for example alkyldimethylamine oxides or alkylaminoethyldimethylamine oxides. Cetyltrimethyl-ammonium salts in particular are advantageously to be used.

### C. Amphoteric Surfactants

Amphoteric surfactants which are advantageously to be used are
acyl-/dialkylethylenediamine, for example sodium acylamphoacetate, disodium acylamphodipropionate, disodium alkylamphodiacetate, sodium acylamphohydroxy-propylsulfonate, disodium acylamphodiacetate and sodium acylamphopropionate,
N-alkylamino acids, for example aminopropyl alkylglutamide, alkylaminopropionic acid, sodium alkylimidodipropionate and lauroamphocarboxyglycinate.

### D. Nonionic Surfactants

Nonionic surfactants which are advantageously to be used are
alcohols,
alkanolamides, such as cocamides MEA/DEA/MIPA,
amine oxides, such as cocoamidopropylamine oxide,
esters which are formed by esterification of carboxylic acids with ethylene oxide, glycerol, sorbitan or other alcohols,
ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and coco-glycoside.
sucrose esters, sucrose ethers
polyglycerol esters, diglycerol esters, monoglycerol esters
methylglucose esters, esters of hydroxy acids

The use of a combination of anionic and/or amphoteric surfactants with one or more nonionic surfactants is furthermore advantageous.

In this context, the surface-active substance(s) can be present in a formulation according to the invention in an amount in the range of from 0.5 to 98 wt. %, based on the total weight of the formulation.

Preferred embodiments and further aspects of the present invention emerge from the attached patent claims and the following examples. Unless stated otherwise, all the data relate to the weight.

### EXAMPLE 1

### Investigations of the Skin-Lightening Action of Styrylresorcinol (Formula 3) In Vivo

The finding that diphenylmethane derivatives of the formula 1 have a particularly good skin-lightening action when they are applied to the skin in formulations having a low content of an oily phase emerges from the following investigations.

An area of skin 1.5 cm² in size on the back of human Asian volunteers (Fitzpatrick skin type III) was treated with emulsions A-F twice daily for 7 days. On day 8 the volunteers were irradiated with UV-A light (4 different doses which each generated a weak, mild, moderate and intense pigmentation). Treatment with emulsions A-F was repeated for a further 14 days. The skin shade (L* values) was determined visually on day 8 and on day 21.

### FORMULATION EXAMPLE 1

| | Substance employed | INCI name | Emulsion A | Emulsion B | Emulsion C |
|---|---|---|---|---|---|
| A. | Dracorin 100 s.e. | PEG 100 Stearate, | 7.0 | 7.0 | 7.0 |
| | | Glyceryl Stearate | | | |
| | PCL Liquid 100 * | Cetearyl Octanoate | 7.5 | 7.5 | 7.5 |
| | Lanette O | Cetearyl Alcohol | 1.0 | 1.0 | 1.0 |
| | Dragoxat 89 * | Ethylhexyl Ethylisononanoate | 2.0 | 2.0 | 2.0 |
| | Neutral oil* | Caprylic/Capric | 6.0 | 6.0 | 6.0 |
| | | Triglyceride | | | |
| | EDTA BD | Disodium EDTA | 0.1 | 0.1 | 0.1 |
| B. | Styrylresorcinol of the | | — | 0.5 | — |
| | formula 3 | | | | |
| | Demineralized water | Water (Aqua) | 70.9 | 70.9 | 69.9 |
| | Kojic acid (Acros) | Kojic Acid | — | — | 1.0 |
| | Glycerol 99% | Glycerin | 4.70 | 4.70 | 4.70 |
| | Dragocid Liquid | Phenoxyethanol, Methyl-, | 0.7 | 0.7 | 0.7 |
| | | Ethyl-, Butyl-, | | | |
| | | Propylparaben, | | | |
| | | Isobutylparaben | | | |
| C. | NaOH soln. 10% | Sodium Hydroxide | 0.1 | 0.1 | 0.1 |
| | strength | | | | |
| | pH | | 5.9 | 5.9 | 6.0 |
| | Content of the oily | | 15.5 | 15.5 | 15.5 |
| | phase (*): | | | | |

### Preparation Instructions:

Heat phases A and B separately to approx. 80° C. Using an Ultra-Turrax stirrer, add phase B to phase A and emulsify. The emulsion is stirred until cold using a blade stirrer, the stirring speed being reduced with decreasing temperature. Adjust the pH of the emulsion to approx. 5.6-5.8 with sodium hydroxide solution.

### Formulation II:

This formulation has a very high water and a low lipid content.

Furthermore, no conventional emulsifier but a surface-modified polymer is employed here. (=aqueous gel cream formulation)

| | Substance | | | | |
|---|---|---|---|---|---|
| | employed | INCI name | Emulsion D | Emulsion E | Emulsion F |
| A. | Neo Pcl wssl. N | Trideceth-9, | 1.5 | 1.5 | 1.5 |
| | | PEG-5 | | | |
| | | Ethylhexanoate | | | |
| | H₂O | Water (Aqua) | 89.35 | 88.85 | 89.85 |
| | Kojic acid (Acros) | Kojic Acid | — | 1.0 | — |
| | Dragocid liquid | Phenoxyethanol, | 0.8 | 0.8 | 0.8 |
| | | Methyl-, Ethyl-, | | | |
| | | Butyl-, | | | |
| | | Propylparaben, | | | |
| | | Isobutylparaben | | | |
| B. | PCL liquid 100 * | Cetearyl Octanoate | 3.0 | 3.0 | 3.0 |
| | Stearic acid | Stearic Acid | 2.0 | 2.0 | 2.0 |
| | Styrylresorcinol of the | | 0.5 | — | — |
| | formula 3 | | | | |
| | Paraffin oil * | Mineral Oil | 2.0 | 2.0 | 2.0 |
| | Abil 350 * | Dimethicone | 0.3 | 0.3 | 0.3 |
| | Pemulen TR1 | Acrylates C10-30 | 0.25 | 0.25 | 0.25 |
| | | Alkyl Acrylate | | | |
| | | Crosspolymer | | | |
| C. | NaOH solution 10% | Sodium Hydroxide | 0.4 | 0.4 | 0.4 |
| | strength | | | | |
| | Total | | 100.0 | 100.0 | 100.0 |
| | pH: | | 5.6 | 5.6 | 5.6 |
| | Content of the oily | | 5.3 | 5.3 | 5.3 |
| | phase (*): | | | | |

### Preparation Instructions:

Heat phases A and B separately to approx. 70-80° C. Using an Ultra-Turrax stirrer, add phase B to phase A and emulsify. Using a blade stirrer, add phase C and stir the emulsion until cold. The pH values are then adjusted to approx. 6.0.

TABLE 1
Result of the visual evaluation on day 21 compared with the particular
placebo and in relation to the starting state before the UV-A irradiation
on day 8:

| Comparison of | Evaluation |
|---|---|
| B (0.5% styrylresorcinol) versus A (placebo) | inactive |
| C (1% kojic acid) versus A (placebo) | active (p > 0.1) |
| D (0.5% styrylresorcinol) versus F (placebo) | active (p = 0.05) |
| E (1% kojic acid) versus F (placebo) | inactive |

Styrylresorcinol is consequently efficient only from the low-fat formulation D (oily phase content 5.3 wt. %), but not from the higher-fat formulation B (oily phase content 15.5 wt. %).

The investigations discussed above clearly show that diphenylmethane derivatives of the formula 1 (wherein R1 to R5 have the meanings given above and that stated above in respect of the preferred meanings of R1 to R5 also applies) have a better action from formulations according to the invention.

### EXAMPLE 2

### Investigation of the Antioxidative Capacity of Styrylresorcinol (Formula 3; CARN 85-27-8; 4-(1-phenylethyl)-1,3-dihydroxybenzene) with the Aid of ABTS Assay

Phenolic compounds often have a very good antioxidative activity. In order to test the extent to which styrylresorcinol (formula 3; CARN 85-27-8; 4-(1-phenylethyl)-1,3-dihydroxybenzene) also has an antioxidative potential, the substance was investigated with the aid of ABTS assay. For qualitative and quantitative estimation of the antioxidative potential, the activity thereof was compared with that of alpha-tocopherol, a highly active and diversely usable antioxidant.

### Description of the ABTS Assay Experiment:

ABTS assay is a cell-free in vitro test for evaluation of antioxidative capacity (literature: Re R, Pellegrini N, Proteggente A, Pannala A, Yang M, Rice-Evans C. 1999. “Antioxidant activity applying an improved ABTS radical cation decolorization assay”; Free Radic. Biol. Med. 26: 1231-7). The assay uses the intrinsic colouration of a solution prepared with the cationic radical 2,2′-azinobis(3-ethylbenzothiazoline-6-sulfonic acid) (ABTS+) and potassium persulfate, which is decolourized by addition of antioxidants (reduction of the cationic radical). This decolouration can be measured photometrically at 734 nm. The test is carried out in 96-well microtitre plates. The antioxidative capacity is expressed in IC₅₀ values (antioxidant concentration at which 50% of the cationic radicals are reduced). All the ABTS test results result from two independent experiments and are presented as means with the associated deviation from the mean.

### Result:

As the ABTS investigations show, the antioxidative capacity of styrylresorcinol (formula 3; CARN 85-27-8,4-(1-phenylethyl)-1,3-dihydroxybenzene) is greater than the antioxidative capacity of the reference substance alpha-tocopherol by about the factor 2, from which it can be seen that styrylresorcinol, alongside its very good skin-lightening and senile keratosis-reducing activity, can also be employed in an outstanding manner as an antioxidant in cosmetic and pharmaceutical products.

TABLE 2
Antioxidative capacity of styrylresorcinol (formula 3; CARN 85-27-8; 4-(1-phenylethyl)-1,3-
dihydroxybenzene) and alpha-tocopherol; IC₅₀ values determined by means of the ABTS method.

| | | IC50 | Standard |
|---|---|---|---|
| Substance | Structural formula | (mean) | deviation |
| Styrylresorcinol (formula 3; CARN 85-27-8; 4-(1- phenylethyl)-1,3- dihydroxybenzene) | 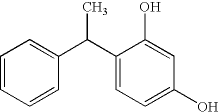 | 0.0134 | 0.0034 |
| alpha-Tocopherol (reference substance) | 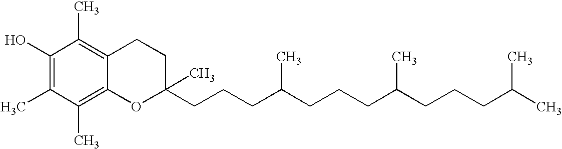 | 0.0226 | 0.0034 |

### EXAMPLE 3

### Influence of the Content of Oily Phase on the Antioxidative Capacity of Styrylresorcinol (Formula 3; CARN 85-27-8; 4-(1-phenylethyl)-1,3-dihydroxybenzene) with the Aid of ABTS Assay

In order to test whether the antioxidative activity of styrylresorcinol (formula 3; CARN 85-27-8; 4-(1-phenylethyl)-1,3-dihydroxybenzene) is adversely influenced by the presence of significant amounts of oily phase, in a further experiment the antioxidative capacity of two products comprising 30 percent by weight of 2-ethylhexyl isononanoate (INCI Ethylhexyl Isononanoate; product A) and, respectively, 5 percent by weight of 2-ethylhexyl isononanoate (INCI Ethylhexyl Isononanoate; product B) was determined with the aid of ABTS assay. The results of the investigations are summarized in Table 3.

TABLE 3
Influence of the content of a lipophilic oil constituent (INCI: Ethylhexyl
Isononanoate) on the antioxidative capacity of styrylresorcinol (formula 3; CARN
85-27-8; 4-(1-phenylethyl)-1,3-dihydroxy-benzene)

| | Product | | |
|---|---|---|---|
| | Product A comprising | | |
| | 0.01 mg/ml | Product B comprising | |
| | styrylresorcinol (formula | 0.01 mg/ml | |
| | 3; CARN 85-27-8; 4-(1- | styrylresorcinol (formula | Product C comprising |
| | phenylethyl)-1,3- | 3; CARN 85-27-8; 4-(1- | 0.01 mg/ml |
| | dihydroxybenzene) and | phenylethyl)-1,3- | styrylresorcinol (formula |
| | 300 mg/ml 2-ethylhexyl | dihydroxybenzene) and | 3; CARN 85-27-8; 4-(1- |
| | isononanoate (ethanolic | 50 mg/ml 2-ethylhexyl | phenylethyl)-1,3- |
| | solution of high oil | isononanoate (ethanolic | dihydroxybenzene) |
| | content) | solution of low oil content) | reference sample (oil- |
| | (30 percent by weight) | (5 percent by weight) | free) |
| Antioxidative | 79.4 | 91.3 | 92.9 |
| capacity | | | |

As the investigations show by way of example, the addition of 30% of oily phase to an ethanolic solution containing 0.01% styrylresorcinol (antioxidative capacity: 79.4%) has the effect of a significant deterioration in the antioxidative capacity by 13.5% compared with the oil-free reference sample containing only 0.01% styrylresorcinol (antioxidative capacity: 92.9%). In a mixture according to the invention comprising only 5% of oily phase (antioxidative capacity: 91.35), on the other hand, only a slight reduction in the antioxidative capacity by 1.6% was determined. The results thus clearly show that products having an oil content of <12% are also advantageous in respect of the antioxidative capacity of styrylresorcinol.

### EXAMPLE 4

### Examples of Formulations According to the Invention Having a Low Content of Oily Phase and which, in Addition to Diphenylmethane Derivatives of the Formula 1, Comprise Further Skin- and Hair-Lightening and Senile Keratosis-Reducing Active Compounds

Cosmetic formulations which show particularly good results in human in vivo use since they have a content of oily phase which is reduced according to the invention are listed by way of example in the table. An improvement in the activity of the formulations is moreover also achieved by the combination of diphenylmethane derivatives of the formula 1 with further skin-lightening and senile keratosis-reducing active compounds.

Preferred embodiments of the present invention emerge from the following examples and the attached patent claims:

Formulation 1: “Oil-in-water” emulsion with UV-A/B-broadband protection
Formulation 2: “Oil-in-water” emulsion with UV-A/B-broadband protection
Formulation 3: Sun spray with UV-A/B-broadband protection with low oil content
Formulation 4: Skin-lightening balm with UV-A/UV-B protection
Formulation 5: Skin-lightening aerosol foam with UV-B/UV-A protection
Formulation 6: Skin-lightening non-aerosol foam
Formulation 7: Shampoo with skin-lightening properties
Formulation 8: Skin-lightening hair conditioner with UV-B/UV-A protection
Formulation 9: Skin-lightening moisturizing cream O/W
Formulation 10: Skin-lightening face cream O/W

| RAW MATERIAL NAME | | | | | | | | | | | |
|---|---|---|---|---|---|---|---|---|---|---|---|
| (MANUFACTURER) | INCI | 1 | 2 | 3 | 4 | 5 | 6 | 7 | 8 | 9 | 10 |
| Skin lightener | | | | | | | | | | | |
| 4-(1-Phenylethyl)- | | 5.0 | 0.05 | 0.2 | 1.0 | 0.5 | 0.1 | 0.5 | 0.2 | | 0.5 |
| 1,3-benzenediol | | | | | | | | | | | |
| 4-Butylresorcinol | | | 0.05 | | | | | | | 1.0 | |
| beta-Arbutin | Arbutin | | | | 0.5 | | | | | 0.2 | |
| Kojic acid | Kojic Acid | | 0.5 | | | | | | | | 1.0 |
| Liquorice extract | | | | | | | 2.0 | | | | |
| Mg ascorbyl | Magnesium Ascorbyl- | | | | | | | | | 3.0 | |
| phosphate | phosphate | | | | | | | | | | |
| Niacinamide | | | | 1.0 | | | | | | | |
| Soya extract | | | 1.0 | | | | | | | | 1.0 |
| Oil components | | | | | | | | | | | |
| Abil 100 ® | Dimethicone | 1.0 | 0.3 | | | | 1.0 | | | 2.0 | 0.3 |
| (Goldschmidt) | | | | | | | | | | | |
| Cetiol OE (Cognis) | Dicaprylyl Ether | | | | | | 3.0 | | | | |
| Corapan TQ ® | Diethylhexyl 2,6- | | 2.0 | | | | | | | | |
| (Symrise) | Naphthalate | | | | | | | | | | |
| Dragoxat EH | Ethylhexyl | | | 1.0 | 1.0 | | | | | | |
| (Symrise) | Ethylhexanoate | | | | | | | | | | |
| Isoadipate (Symrise) | Diisopropyl Adipate | | | | 1.0 | | | | | | |
| Isopropyl myristate | Isopropyl Myristate | | | | | | | | | | 4.0 |
| (Symrise) | | | | | | | | | | | |
| Isodragol (Symrise) | Triisononanoin | | | | | | | | | 6.0 | |
| Neutral oil (Symrise) | Caprylic/Capric/ | 4.0 | 2.0 | | | 2.0 | | | | | |
| | Triglyceride | | | | | | | | | | |
| Paraffin oil | Mineral Oil | | | | | | | | | | 4.0 |
| PCL Liquid 100 | Cetearyl Octanoate | 4.0 | 3.0 | | | | | | 3.0 | | |
| (Symrise) | | | | | | | | | | | |
| Tegosoft TN ® | C12-C15 Alkyl Benzoate | | | | 4.0 | 2.0 | | | | | |
| (Goldschmidt) | | | | | | | | | | | |
| Further ingredients | | | | | | | | | | | |
| Arlypon F | Laureth-2 | | | | | | | 2.0 | | | |
| alpha-Bisabolol | Bisabolol | | 0.1 | | 0.2 | 0.1 | 0.1 | 0.1 | | | |
| (Symrise) | | | | | | | | | | | |
| 1,3-Butylene glycol | 1,3-Butylene Glycol | | 3.0 | | | | | | | | |
| Carbopol 2050 ® | Carbomer | | 0.2 | | 0.5 | 0.1 | | | | | |
| (B. F. Goodrich) | | | | | | | | | | | |
| Ceramid Bio 391 | Ceramide | 0.3 | | | | | | | | | 0.1 |
| (Symrise) | | | | | | | | | | | |
| Citric Acid | Citric Acid | | | | | | | | 0.1 | 0.3 | |
| Copherol 1250 ® | Tocopherol Acetate | | 0.5 | | 0.5 | 0.5 | 0.5 | | | | |
| (Cognis) | | | | | | | | | | | |
| Crinipan ® AD | Climbazole | | | | | | | 0.5 | | | |
| (Symrise) | | | | | | | | | | | |
| Dehyquart SP | Quaternium-52 | | | | | | | | 0.5 | | |
| Dehyton K | Cocamidopropyl Betaine | | | | | | | 12.0 | | | |
| Dow Corning ® 193 | Dimethicone Polyol | | | 1.0 | | | | | | | |
| (Dow Corning) | | | | | | | | | | | |
| D-Panthenol (BASF) | Panthenol | | | 0.5 | | | 0.5 | 0.4 | | | |
| Dragocid Liquid | Phenoxyethanol (and) | 0.8 | | 0.8 | 0.8 | 0.8 | 0.8 | 0.5 | 0.8 | 0.8 | |
| (Symrise) | Methylparaben (and) | | | | | | | | | | |
| | Ethylparaben (and) | | | | | | | | | | |
| | Butylparaben (and) | | | | | | | | | | |
| | Propylparaben (and) | | | | | | | | | | |
| | Isobutylparaben | | | | | | | | | | |
| Dragophos S | Sodium Dihydroxycetyl | | | | | | | | | 2.0 | |
| (Symrise) | Phosphate | | | | | | | | | | |
| Dracorin CE | Glyceryl Stearate/Citrate | | | | | | | | | 1.0 | |
| Dracorin GMS | Glyceryl Stearate | 2.0 | 2.0 | | | 2.0 | | | | 2.0 | 3.0 |
| (Symrise) | | | | | | | | | | | |
| Dracorin 100 s.e. P | Glyceryl Stearate, PEG- | 3.0 | | | | | | | | | 8.0 |
| (Symrise) | 100 Stearate | | | | | | | | | | |
| Edeta BD ® (BASF) | Disodium EDTA | 0.1 | 0.1 | | 0.1 | 0.1 | 0.1 | | | | |
| Emulgin B2 ® | Ceteareth-20 | 1.0 | | | | | | | 0.7 | | |
| (Cognis) | | | | | | | | | | | |
| Emulsiphos | Potassium | | 1.5 | | | 1.5 | | | | | |
| (Symrise) | Cetylphosphate, | | | | | | | | | | |
| | Hydrogenated Palm | | | | | | | | | | |
| | Glycerides | | | | | | | | | | |
| Ethanol (96%) | Ethyl Alcohol | | | 13.0 | 5.0 | | | | | | |
| Extrapon Aloe Vera | | | | 1.0 | | | | | | | |
| (Symrise) | | | | | | | | | | | |
| Extrapon Kamille | | | | 1.0 | | | | | | | |
| (Symrise) | | | | | | | | | | | |
| Extrapon Hamamelis | | | | 1.0 | | | | | | | |
| (Symrise) | | | | | | | | | | | |
| Frescolat ML | Menthyl Lactate | | | | 0.5 | | | | | | |
| Glycerol 99% | Glycerin | 3.0 | | 4.5 | | 3.0 | 4.0 | | | | |
| Hydrolite-5 | Pentylene Glycol | | | | 4.5 | | 5.0 | | | 3.0 | |
| (Symrise) | | | | | | | | | | | |
| Keltrol T ® (Kelco) | Xanthan Gum | | 0.2 | 0.2 | 0.3 | | | | | | |
| Lanette E ® (Cognis) | Sodium Cetearyl Sulfate | | 0.7 | | | | | | | | |
| Lanette O ® (Cognis) | Cetaeryl Alcohol | 1.1 | | | | | | | 2.5 | | |
| Lanette 16 ® | Cetyl Alcohol | | 1.2 | | | 0.5 | | | | | 2.0 |
| (Cognis) | | | | | | | | | | | |
| Lanette 18 (Care | Stearyl Alcohol | | | | | | | | | 4.5 | |
| Chemicals) | | | | | | | | | | | |
| Lara Care A-200 | Galactoarabinan | | | | | | 0.2 | | | | |
| (Rahn) | | | | | | | | | | | |
| NaOH 10% aq. | Sodium Hydroxide | | 2.8 | | 2.2 | 2.9 | 0.6 | | | | 0.2 |
| solution | | | | | | | | | | | |
| Natrosol 250 HHR | Hydroxymethyl Cellulose | 0.3 | | | | | | | | | |
| (Aqualon) | | | | | | | | | | | |
| Neo Heliopan ® AP | Disodium Phenyl- | | 22.0 | | | | | | | | |
| (Symrise), 15% as | dibenzimidazole Tetra- | | | | | | | | | | |
| sodium salt | sulfonate | | | | | | | | | | |
| Neo Heliopan ® AP | Disodium Phenyl- | | | 22.0 | | | | | | | |
| (Symrise), 10% aq. | dibenzimidazole Tetra- | | | | | | | | | | |
| solution neutralized | sulphonate | | | | | | | | | | |
| with NaOH | | | | | | | | | | | |
| Neo Heliopan ® AV | Ethylhexyl Methoxy- | | | | 5.0 | 6.0 | 2.0 | | | | |
| (Symrise) | cinnamate | | | | | | | | | | |
| Neo Heliopan ® BB | Benzophenone-3 | 1.0 | | | | | | | | | |
| (Symrise) | | | | | | | | | | | |
| Neo Heliopan ® 303 | Octocrylene | 7.0 | | | | | | | | | |
| (Symrise) | | | | | | | | | | | |
| Neo Heliopan ® 357 | Butyl Methoxydibenzoyl- | | 2.0 | | 1.5 | 1.5 | 1.5 | 0.5 | 0.5 | | |
| (Symrise) | methane | | | | | | | | | | |
| Neo Heliopan ® | Isoamyl p- | | | | 5.0 | | 6.0 | | 2.0 | | |
| E 1000 (Symrise) | Methoxycinnamate | | | | | | | | | | |
| Neo Heliopan ® | Homosalate | | 5.0 | | | | | | | | |
| HMS (Symrise) | | | | | | | | | | | |
| Neo Heliopan ® | Phenylbenzimidazole | | | 33.3 | 10.0 | 13.3 | | 3.3 | | | |
| Hydro | Sulfonic Acid | | | | | | | | | | |
| (15% aq. solution | | | | | | | | | | | |
| neutralized with | | | | | | | | | | | |
| NaOH) (Symrise) | | | | | | | | | | | |
| Neo Heliopan ® MA | Menthyl Anthranilate | 3.0 | | | | | | | | | |
| (Symrise) | | | | | | | | | | | |
| Neo Heliopan ® | 4-Methylbenzylidene | | | | 2.0 | 4.0 | 3.0 | | | | |
| MBC (Symrise) | Camphor | | | | | | | | | | |
| Neo Heliopan ® OS | Ethylhexyl Salicylate | 1.0 | | | | | | | | | |
| (Symrise) | | | | | | | | | | | |
| Neo PCL wssl. N | Trideceth-9, PEG-5 | | | 1.0 | 1.5 | | | 1.5 | | | |
| (Symrise) | Ethylhexanoate | | | | | | | | | | |
| Perfume oil | Perfume (Fragrance) | 0.3 | 0.3 | 0.3 | 0.3 | 0.4 | 0.2 | 0.5 | 0.4 | 0.3 | 0.3 |
| (Symrise) | | | | | | | | | | | |
| Pemulen TR 2 | Acrylates/C10-30 Alkyl | | | | | | 0.2 | | | | |
| (Novion) | Acrylate Crosspolymer | | | | | | | | | | |
| Polymer JR 400 | Polyquaternium-10 | | | | | | | 0.4 | | | |
| 1,2-Propylene glycol | Propylene Glycol | | | | | | | | | | 5.0 |
| Simagel M | Quaternium-18 Hectorite | 1.0 | | | | | | | | | |
| Solubilizer (Symrise) | PEG 40 Hydrogenated | | | | | | | 3.0 | | | |
| | Castor Oil, Trideceth-9, | | | | | | | | | | |
| | Propylene Glycol, Water | | | | | | | | | | |
| Symdiol 68 | 1,2-Hexanediol, | | 0.5 | | | | | | | | 1.0 |
| | Caprylylglycol | | | | | | | | | | |
| Texapon N 70 | Sodium Laureth Sulfate | | | | | | 0.5 | | | | |
| (Cognis) | | | | | | | | | | | |
| Texapon NSO BZ | Sodium Laureth Sulfate | | | | | | | 27.0 | | | |
| (Cognis) | | | | | | | | | | | |
| Zink Oxide neutral | Zinc Oxide | 5.0 | | | | | | | | | |
| (Symrise) | | | | | | | | | | | |
| Veegum ultra ® | Magnesium Aluminium | 1.0 | | | | | | | | | |
| (Vanderbilt) | Sulfate | | | | | | | | | | |
| Vitamin A Palmitate | Retinyl Palmitate | | | | | 0.1 | | | | 0.1 | |
| Witch Hazel | Hamamelis Virginiana | | | | | | | 1.0 | | | |
| Distillate (Symrise) | (Witch Hazel) | | | | | | | | | | |
| Water, dist. | Aqua (Water) | ad | ad | ad | ad | ad | ad | ad | ad | ad | ad |
| | | 100 | 100 | 100 | 100 | 100 | 100 | 100 | 100 | 100 | 100 |

### Specific Embodiments

In specific embodiment one, the invention comprises a cosmetic and/or pharmaceutical formulation, comprising a tyrosinase-inhibiting amount of one or more compounds of the formula 1:

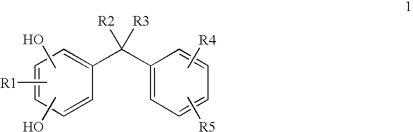

wherein:

### R1 is

hydrogen,
methyl,
straight-chain or branched, saturated or unsaturated alkyl having 2-4 C atoms,
OH or
halogen,

### R2 is

hydrogen,
methyl or
straight-chain or branched, saturated or unsaturated alkyl having 2-5 C atoms,

### R3 is

methyl or
straight-chain or branched, saturated or unsaturated alkyl having 2-5 C atoms,
   and
   R4 and R5 are, independently of one another,
hydrogen,
methyl,
straight-chain or branched, saturated or unsaturated alkyl having 2-5 C atoms,
OH or
   halogen
   and an oily phase, wherein the content of the oily phase in the formulation is 0.05 to 12 wt. %.

In specific embodiment two, the invention comprises a formulation according to specific embodiment one, wherein the compound of the formula 1 is styrylresorcinol.

In specific embodiment three, the invention comprises a formulation according to specific embodiment one or two, having a water content of from 25 to 95 wt. %, preferably from 40 to 90 wt. %, in each case based on the total weight of the formulation.

In specific embodiment four, the invention comprises a formulation according to specific embodiment one, wherein this is in the form of an O/W emulsion.

In specific embodiment five, the invention comprises a formulation according to one of the preceding specific embodiments, furthermore comprising at least one UV filter, preferably at least in an amount which is capable of preventing discolouration of the formulation caused by (sun)light.

In specific embodiment six, the invention comprises a formulation according to one of the preceding specific embodiments, furthermore comprising a total amount of UV filters and/or inorganic pigments such that the formulation according to the invention has a sunscreen factor of greater than or equal to 2, preferably greater than or equal to 5.

In specific embodiment seven, the invention comprises a formulation according to one of the preceding specific formulations, furthermore comprising a further skin- and/or hair-lightening active compound, preferably in an amount which has a skin- or hair-lightening action.

In specific embodiment eight, the invention comprises a formulation according to one of the preceding specific embodiments, furthermore comprising a cooling active compound in an amount sufficient to achieve a skin-cooling effect.

In specific embodiment nine, the invention comprises a formulation according to one of the preceding specific embodiments, furthermore comprising one or more compounds for care and/or cleansing of (a) skin and/or (b) hair.

In specific embodiment ten, the invention comprises a formulation according to one of the preceding specific embodiments, furthermore comprising a sensorially active amount of one or more odoriferous substances.

In specific embodiment eleven, the invention comprises a method for lightening skin and/or hair and/or for reducing senile keratosis, comprising the step:
application of a formulation according to one of the preceding specific embodiments to skin and/or hair.

In specific embodiment twelve, the invention comprises a process for the preparation of a formulation for lightening skin and/or hair and/or for reducing senile keratosis, comprising the step:
mixing of a compound of the formula 1 according to specific embodiment one with an oily phase, wherein the weight content of the oily phase in the finished formulation is 0.05 to 12 wt. %.

It is claimed:

1. A cosmetic and/or pharmaceutical formulation, comprising a tyrosinase-inhibiting amount of one or more compounds of the formula 1: 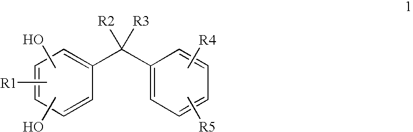
wherein:
R1 is
hydrogen,
methyl,
straight-chain or branched, saturated or unsaturated alkyl having 2-4 C atoms,
OH or
halogen,
R2 is
hydrogen,
methyl or
straight-chain or branched, saturated or unsaturated alkyl having 2-5 C atoms,
R3 is
methyl or
straight-chain or branched, saturated or unsaturated alkyl having 2-5 C atoms,
and
R4 and R5 are, independently of one another,
hydrogen,
methyl,
straight-chain or branched, saturated or unsaturated alkyl having 2-5 C atoms,
OH or
halogen
and an oily phase, wherein the content of the oily phase in the formulation is 0.05 to 12 wt. %.

2. The formulation according to claim 1, wherein the compound of the formula 1 is styrylresorcinol.

3. The formulation according to claim 1, further comprising a water content of from 25 to 95 wt. %, based on the total weight of the formulation.

4. The formulation according to claim 1, wherein the formulation is in the form of an O/W emulsion.

5. The formulation according to claim 1, further comprising at least one UV filter.

6. The formulation according to claim 1, further comprising a total amount of UV filters and/or inorganic pigments such that the formulation according to the invention has a sunscreen factor of greater than or equal to 2.

7. The formulation according to claim 1, further comprising a further skin- and/or hair-lightening active compound.

8. The formulation according to claim 1, further comprising a cooling active compound in an amount sufficient to achieve a skin-cooling effect.

9. The formulation according to claim 1, further comprising one or more compounds for care and/or cleansing of (a) skin and/or (b) hair.

10. The formulation according to claim 1, further comprising a sensorially active amount of one or more odoriferous substances.

11. A method for lightening skin and/or hair and/or for reducing senile keratosis, comprising the step:
application of a formulation according to claim 1 to skin and/or hair.

12. A process for the preparation of a formulation for lightening skin and/or hair and/or for reducing senile keratosis, comprising the step:
mixing of a compound of the formula 1 according to claim 1 with an oily phase, wherein the weight content of the oily phase in the finished formulation is 0.05 to 12 wt. %.

13. The formulation according to claim 1, wherein the water content is from 40 to 90 wt. %.

14. The formulation according to claim 1, wherein the at least one UV filter is present at least in an amount which prevents discolouration of the formulation caused by (sun)light.

15. The formulation according to claim 1, wherein the formulation has a sunscreen factor of greater than or equal to 5.

16. The formulation according to claim 1, wherein the further skin- and/or hair-lightening active compound is present in an amount which has a skin- or hair-lightening action.
